SEQUENCE LISTING (24 sequences)

SEQ ID NO: 1 - DNA, 1480 bp, synthetic construct
  source 1..1480: mol_type = other DNA
aatgctccttttggtcacatctttgttgctttgcgaactgcctcatccagcgttcttgcttataccagatatccagatgacacagacgacctccagcctctcagcttccttgggggatagggtaaccatttcttgtagagcttcccaagatatcagcaagtaccttaattggtatcagcagaagcccgatggaactgtgaagttgctgatttaccacacatctagattgcactccggagtaccgagccgcttcagcggctcaggatcagggactgactactcactgactatcagcaatcttgaacaagaggacattgcgacttatttttgccaacagggaaacacactgccttatacctttggaggcggtacaaaacttgaaataacaggatctacctctggatccggtaaaccagggtccggagaaggatctaccaaaggtgaagtcaagctccaagaaagcggaccaggccttgttgcacccagccaatctttgtcagttacgtgcacagtgtcaggcgtatccttgccggactacggcgtgtcatggatcagacaaccccctagaaaaggtctggagtggctgggagtgatatggggaagtgaaactacctattataatagtgcgcttaaatcacggctcacaattataaaggacaattctaagtcacaagtcttcttgaagatgaactctcttcaaacggacgacaccgccatttactactgcgctaagcattactattatggggggtcctatgcaatggattattggggccaagggacctctgtaacggtgagtagcgcagctgccacaacgaccccggcacctcgaccaccaaccccagcgcctacaattgcgtcccaacccttgagccttaggccagaggcatgccggcccgcggctggcggagctgtgcatactcgcggattggattttgcttgtgacatctacatttgggccccactcgctgggacctgtggggtcttgcttctgtccttggtaataactttgtactgcaagcgggggcggaaaaaactcctctatattttcaaacaaccttttatgagaccagttcagacaacgcaggaagaggatggctgctcatgtcggtttcccgaggaggaagaaggtggttgtgaactgcgagttaagttctccagatcagcagatgccccggcatatcagcaggggcaaaaccaactctataatgagttgaatctcggcagaagggaggaatatgatgtgctggacaaaagaagaggcagggacccggagatgggagggaaacctaggcgaaaaaaccctcaagagggactttataacgaacttcagaaagacaagatggcagaggcgtacagtgaaattggtatgaaaggtgaaagacgaagggggaagggacacgatggcttgtaccaagggctcagcacagcaacgaaggacacatacgatgcgttgcacatgcaagcactcccgccccgc

SEQ ID NO: 2 - protein, 493 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - DNA, 685 bp, synthetic construct
  source 1..685: mol_type = other DNA
aatgattataggcggacatgaagcaaagccgcactcccggccgtacatggcctacttgatgatttgggatcaaaaatcccttaaaaggtgcggaggcttcctgattcgcgatgattttgtactgactgccgcccactgctggggcagttctataaacgtcactcttggtgcacataacatcaaagagcaggaacctacacaacaattcatacccgtcaagcgaccaattccccatcctgcatataatccgaaaaacttttctaatgacataatgctgctccaacttgagaggaaggcaaagcggacgcgcgccgttcaacccttgcgactcccgtcaaacaaagcccaggtcaaaccaggacaaacgtgctcagttgcgggttggggtcaaacagcgcccctcggcaagcactcacacactcttcaagaagtaaaaatgacggttcaagaagatcgcaaatgcgagtctgacctgaggcattattacgattccaccatagagctttgtgtgggtgatccagagattaaaaagacttcattcaaaggagacagtggtggacctcttgtgtgcaataaagtcgctcaagggatcgtttcatacggtagaaataacggcatgcctcctcgcgcttgtactaaagtgtcatccttcgttcattggatcaaaaaaacaatgaaaagatac

SEQ ID NO: 4 - protein, 228 aa, synthetic construct
MIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRY

SEQ ID NO: 5 - DNA, 312 bp, synthetic construct
  source 1..312: mol_type = other DNA
gaagtacaactcgtcgagagcggcggcggccccgtgcagccaggaggttcattgcggctgtcatgtactgggagtggattcttcggtatgtgggtgaggcagccagggaaggggcttgaatgggtatcaatcggcacctatgcggacagtgtgaaaggccgattcagcagggacaacgccaagaataccttgtatcttcaaatgaatcttgaagatactgcggtttattgcgctaagccggtcacaatacttttcgggacgtggaagcggtacgactactggggacaagggacgcaggtcacagttagttct

SEQ ID NO: 6 - protein, 104 aa, synthetic construct
EVQLVESGGGPVQPGGSLRLSCTGSGFFGMWVRQPGKGLEWVSIGTYADSVKGRFSRDNAKNTLYLQMNLEDTAVYCAKPVTILFGTWKRYDYWGQGTQVTVSS

SEQ ID NO: 7 - DNA, 1326 bp, synthetic construct
  source 1..1326: mol_type = other DNA
atggccggagagggagatcaacaggatgcggcgcataacatgggaaaccacctgcctttgcttccagccgagtcagaggaggaagacgagatggaagtagaggaccaggattcaaaagaggctaagaaaccaaacatcatcaacttcgacaccagtctgcctactagccacacatacctgggggctgacatggaagagtttcacgggcggacacttcacgacgacgatagctgtcaagttatccctgtcctgccccaggtcatgatgatccttattcccgggcaaaccctccccctgcaactcttccacccacaagaggttagcatggtaagaaacctcatacagaaggatagaacattcgcggtgttggcttattcaaatgtacaggagagggaagcacaattcggtacgactgccgaaatatacgcatatagagaggagcaggattttggcatcgaaatagtaaaggttaaggccatcggtcgccaacgatttaaggtactcgaactgaggacccagagtgacgggattcaacaggccaaagtccagatactgcccgagtgtgtgttgccttcaactatgtcagccgttcaacttgaatctcttaacaagtgccaaatctttccgtccaaaccagtaagccgcgaagaccaatgttcttataagtggtggcaaaaatatcagaagcgaaagtttcattgcgctaatcttacgtcatggccgcggtggctttatagtctttacgacgcggaaactctgatggacaggattaagaaacagctgcgagagtgggacgagaatcttaaagatgattctctcccttcaaatcctattgactttagttacagagtcgccgcttgtttgcccatcgacgatgtgctgagaatccagctgttgaaaatcggttcagccatccagaggttgcgatgtgaacttgatataatgaataaatgcactagtctctgctgtaagcaatgccaggagacagaaattacaacgaaaaatgaaattttttcccttagcctgtgtgggcctatggccgcttatgtgaacccgcacggttacgtgcacgagactctgaccgtgtacaaggcctgcaaccttaacttgattggtcgaccatcaacagagcacagctggtttccggggtacgcttggaccgtggcacaatgtaagatctgcgcgagtcatattgggtggaagttcacagcgactaagaaggatatgagtccgcagaagttctggggcttgacacgctctgctcttctgccaaccatcccagatacggaagatgaaattagcccagacaaggtaattctctgcctc

SEQ ID NO: 8 - protein, 442 aa, synthetic construct [sequence omitted]

SEQ ID NO: 9 - DNA, 2433 bp, synthetic construct
  source 1..2433: mol_type = other DNA
atgattattggtggacacgaagccaagccacacagccgaccatacatggcatatcttatgatctgggaccagaagagcctcaagcggtgcggcggattcttgatcagagacgactttgtgcttacagctgctcattgctggggctccagcatcaatgtcacactcggcgcccacaatatcaaagaacaagaaccaacgcaacagtttatcccggtcaaacgacccataccccatcctgcctacaacccgaagaacttcagcaatgacataatgctgctccaactcgagcggaaggcgaaaaggactcgggccgtccaaccactcagattgccctccaataaagctcaggtaaaaccaggccaaacgtgctctgtcgcaggctgggggcaaacagcaccccttggtaagcatagtcatactttgcaagaagttaaaatgacagtacaggaagatcgaaaatgtgaatctgaccttcgccattattacgattccaccattgagctgtgtgtaggagatccagaaatcaagaagactagttttaagggcgattcaggaggaccgctggtgtgcaataaagtggcccagggaatcgtttcttacggaaggaacaacggaatgcctccgcgggcatgcactaaggtgtcttcctttgtacattggattaaaaagacaatgaaacgctacggtggcggcggatccgggggcggcggttctggagggggcggctcagaagtacagcttgtagaatcaggtggcgggccggtacagcccggggggagtcttcgactgtcttgcacggggtctggcttcttcggcatgtgggtcaggcagccagggaaagggcttgaatgggtgagcataggaacttatgcggacagtgtcaaaggaagattcagccgagataacgcgaagaacactttgtacctccaaatgaatctggaagacaccgccgtgtactgcgccaagccggtcactatcctgttcgggacctggaagaggtatgattactggggacaagggacacaagtaaccgtcagcagcggcggcgggggaagcggtggtggtgggtccggaggtggcggatccatggctggagagggagatcaacaagacgcagctcataatatgggaaatcatctccccctgctgccggcagaaagcgaggaagaggacgagatggaggtggaggatcaagattccaaagaggctaagaagccaaatatcatcaacttcgatacttccctgcctacctcacatacttatcttggtgctgatatggaggagttccatggtcgcaccttgcatgatgatgattcttgccaggtaattcctgtgttgccgcaggtgatgatgatacttatccctggtcaaacgttgccgttgcaacttttccacccacaggaagtttctatggtcaggaatctcatccagaaggacagaacgtttgcggttttggcgtatagtaacgtacaagagagggaagcacaattcggtacgactgccgagatatatgcttatcgcgaggaacaggacttcggaattgaaatagtaaaggtaaaagcaattgggcgccaacggtttaaagtattggagctcaggacgcaaagtgatggcatccagcaggcaaaggtgcagatattgcccgaatgtgttcttccctctacgatgtctgccgtgcagttggaatccctgaacaagtgtcaaattttcccctcaaaaccagtcagcagggaggatcaatgtagttataagtggtggcagaaatatcagaagcgaaagttccactgcgccaacctgacaagttggccacggtggctgtattctctgtatgatgcggagacactgatggacagaatcaaaaaacaacttagggaatgggatgaaaacctgaaagacgactcccttccgtccaacccaatagactttagttatcgcgtcgctgcgtgcctccctatcgatgacgttcttagaatccaactcctcaagatcggcagcgctattcagaggctgcggtgtgagctggatataatgaataaatgcacgagtctttgttgtaaacagtgccaagaaacggagataaccacaaagaatgaaatattcagtctgtctctttgcgggccgatggcagcatatgtgaatcctcatgggtacgtgcacgagacgctgacagtctacaaggcgtgcaatctcaacctgatcggaaggccatccacggaacacagttggtttcccggctatgcatggacagtagcgcagtgtaagatttgtgcaagccatataggttggaagttcaccgcgacgaagaaggacatgtctcctcagaaattctggggcctcacacgctctgcacttctcccgacaataccggatacggaggacgagatctcccctgacaaagtgatcctctgcctcgattacgacgatgatgataag

SEQ ID NO: 10 - protein, 811 aa, synthetic construct
MIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRYGGGGSGGGGSGGGGSEVQLVESGGGPVQPGGSLRLSCTGSGFFGMWVRQPGKGLEWVSIGTYADSVKGRFSRDNAKNTLYLQMNLEDTAVYCAKPVTILFGTWKRYDYWGQGTQVTVSSGGGGSGGGGSGGGGSMAGEGDQQDAAHNMGNHLPLLPAESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTMSAVQLESLNKCQIFPSKPVSREDQCSYKWWQKYQKRKFHCANLTSWPRWLYSLYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCLDYDDDDK

SEQ ID NO: 11 - DNA, 794 bp, synthetic construct
  source 1..794: mol_type = other DNA
atgactaagttttcttccttttctctgtttttcctaatagttggggcttatatgactcatgtgtgtttcaatatggaaattattggagggaaagaagtgtcacctcattccaggccatttatggcctccatccagtatggcggacatcacgtttgtggaggtgttctgattgatccacagtgggtgctgacagcagcccactgccaatatcggtttaccaaaggccagtctcccactgtggttttaggcgcacactctctctcaaagaatgaggcctccaaacaaacactggagatcaaaaaatttataccattctcaagagttacatcagatcctcaatcaaatgatatcatgctggttaagcttcaaacagccgcaaaactcaataaacatgtcaagatgctccacataagatccaaaacctctcttagatctggaaccaaatgcaaggttactggctggggagccaccgatccagattcattaagaccttctgacaccctgcgagaagtcactgttactgtcctaagtcgaaaactttgcaacagccaaagttactacaacggcgacccttttatcaccaaagacatggtctgtgcaggagatgccaaaggccagaaggattcctgtaagggtgactcagggggccccttgatctgtaaaggtgtcttccacgctatagtctctggaggtcatgaatgtggtgttgccacaaagcctggaatctacaccctgttaaccaagaaataccagacttggatcaaaagcaaccttgtcccgcctcatacaaatta

SEQ ID NO: 12 - protein, 264 aa, synthetic construct
MTKFSSFSLFFLIVGAYMTHVCFNMEIIGGKEVSPHSRPFMASIQYGGHHVCGGVLIDPQWVLTAAHCQYRFTKGQSPTVVLGAHSLSKNEASKQTLEIKKFIPFSRVTSDPQSNDIMLVKLQTAAKLNKHVKMLHIRSKTSLRSGTKCKVTGWGATDPDSLRPSDTLREVTVTVLSRKLCNSQSYYNGDPFITKDMVCAGDAKGQKDSCKGDSGGPLICKGVFHAIVSGGHECGVATKPGIYTLLTKKYQTWIKSNLVPPHTN

SEQ ID NO: 13 - DNA, 976 bp, synthetic construct
  source 1..976: mol_type = other DNA
atggcccccaagaagaagaggaaggtgggcatccacggggtacctatggtggacttgaggacactcggttattcgcaacagcaacaggagaaaatcaagcctaaggtcaggagcaccgtcgcgcaacaccacgaggcgcttgtggggcatggcttcactcatgcgcatattgtcgcgctttcacagcaccctgcggcgcttgggacggtggctgtcaaataccaagatatgattgcggccctgcccgaagccacgcacgaggcaattgtaggggtcggtaaacagtggtcgggagcgcgagcacttgaggcgctgctgactgtggcgggtgagcttagggggcctccgctccagctcgacaccgggcagctgctgaagatcgcgaagagagggggagtaacagcggtagaggcagtgcacgcctggcgcaatgcgctcaccggggcccccttgaacctgaccccagaccaggtagtcgcaatcgcgaacaataatgggggaaagcaagccctggaaaccgtgcaaaggttgttgccggtcctttgtcaagaccacggccttacaccggagcaagtcgtggccattgcaaataataacggtggcaaacaggctcttgagacggttcagagacttctcccagttctctgtcaagcccacgggctgactcccgatcaagttgtagcgattgcgtccaacggtggagggaaacaagcattggagactgtccaacggctccttcccgtgttgtgtcaagcccacggtttgacgcctgcacaagtggtcgccatcgccagccatgatggcggtaagcaggcgctggaaacagtacagcgcctgctgcctgtactgtgccaggatcatggactgaccccagaccaggtagtcgcaatcgcgtcacatgacgggggaaagcaagccctggaaaccgtgcaaaggttgttgccggtcctttgtcaagaccacggccttacaccgg

SEQ ID NO: 14 - protein, 325 aa, synthetic construct
MAPKKKRKVGIHGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASHDGGKQALETVQRLLPVLCQDHGLTP

SEQ ID NO: 15 - DNA, 1783 bp, synthetic construct
  source 1..1783: mol_type = other DNA
atgactaagttttcttccttttctctgtttttcctaatagttggggcttatatgactcatgtgtgtttcaatatggaaattattggagggaaagaagtgtcacctcattccaggccatttatggcctccatccagtatggcggacatcacgtttgtggaggtgttctgattgatccacagtgggtgctgacagcagcccactgccaatatcggtttaccaaaggccagtctcccactgtggttttaggcgcacactctctctcaaagaatgaggcctccaaacaaacactggagatcaaaaaatttataccattctcaagagttacatcagatcctcaatcaaatgatatcatgctggttaagcttcaaacagccgcaaaactcaataaacatgtcaagatgctccacataagatccaaaacctctcttagatctggaaccaaatgcaaggttactggctggggagccaccgatccagattcattaagaccttctgacaccctgcgagaagtcactgttactgtcctaagtcgaaaactttgcaacagccaaagttactacaacggcgacccttttatcaccaaagacatggtctgtgcaggagatgccaaaggccagaaggattcctgtaagggtgactcagggggccccttgatctgtaaaggtgtcttccacgctatagtctctggaggtcatgaatgtggtgttgccacaaagcctggaatctacaccctgttaaccaagaaataccagacttggatcaaaagcaaccttgtcccgcctcatacaaatggtggtggtggtagtatggcccccaagaagaagaggaaggtgggcatccacggggtacctatggtggacttgaggacactcggttattcgcaacagcaacaggagaaaatcaagcctaaggtcaggagcaccgtcgcgcaacaccacgaggcgcttgtggggcatggcttcactcatgcgcatattgtcgcgctttcacagcaccctgcggcgcttgggacggtggctgtcaaataccaagatatgattgcggccctgcccgaagccacgcacgaggcaattgtaggggtcggtaaacagtggtcgggagcgcgagcacttgaggcgctgctgactgtggcgggtgagcttagggggcctccgctccagctcgacaccgggcagctgctgaagatcgcgaagagagggggagtaacagcggtagaggcagtgcacgcctggcgcaatgcgctcaccggggcccccttgaacctgaccccagaccaggtagtcgcaatcgcgaacaataatgggggaaagcaagccctggaaaccgtgcaaaggttgttgccggtcctttgtcaagaccacggccttacaccggagcaagtcgtggccattgcaaataataacggtggcaaacaggctcttgagacggttcagagacttctcccagttctctgtcaagcccacgggctgactcccgatcaagttgtagcgattgcgtccaacggtggagggaaacaagcattggagactgtccaacggctccttcccgtgttgtgtcaagcccacggtttgacgcctgcacaagtggtcgccatcgccagccatgatggcggtaagcaggcgctggaaacagtacagcgcctgctgcctgtactgtgccaggatcatggactgaccccagaccaggtagtcgcaatcgcgtcacatgacgggggaaagcaagccctggaaaccgtgcaaaggttgttgccggtcctttgtcaagaccacggccttacaccgg

SEQ ID NO: 16 - protein, 594 aa, synthetic construct
MTKFSSFSLFFLIVGAYMTHVCFNMEIIGGKEVSPHSRPFMASIQYGGHHVCGGVLIDPQWVLTAAHCQYRFTKGQSPTVVLGAHSLSKNEASKQTLEIKKFIPFSRVTSDPQSNDIMLVKLQTAAKLNKHVKMLHIRSKTSLRSGTKCKVTGWGATDPDSLRPSDTLREVTVTVLSRKLCNSQSYYNGDPFITKDMVCAGDAKGQKDSCKGDSGGPLICKGVFHAIVSGGHECGVATKPGIYTLLTKKYQTWIKSNLVPPHTNGGGGSMAPKKKRKVGIHGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASHDGGKQALETVQRLLPVLCQDHGLTP

SEQ ID NO: 17 - DNA, 345 bp, synthetic construct
  source 1..345: mol_type = other DNA
ctggctcgagctcatctgcgggatgaagagaagtcatgcccatgcttggctcaggaaggtccgcagggagacttgctcactaagacccaggagttgggtcgagactatcgcacgtgtctcacaatagtgcagaaactcaagaaaatggtcgacaaaccgacacagcgcagtgtcagtaacgctgctacgcgagtatgtcgaaccgggcggagtcggtggcgggatgtgtgccgaaatttcatgcgaaggtatcaatcccgagtcacgcagggcctggtggccggtgaaaccgcccaacagatttgcgaggacttgcgactctgcataccctccacgggtccactc

SEQ ID NO: 18 - protein, 115 aa, synthetic construct
LARAHLRDEEKSCPCLAQEGPQGDLLTKTQELGRDYRTCLTIVQKLKKMVDKPTQRSVSNAATRVCRTGRSRWRDVCRNFMRRYQSRVTQGLVAGETAQQICEDLRLCIPSTGPL

SEQ ID NO: 19 - DNA, 738 bp, synthetic construct
  source 1..738: mol_type = other DNA
atggctcaagttcaactccagcaatcaggcccaggtcttgttaaaccctcccagaccctctctctcacctgtgctatttccggcgactctgtcagtagcaactccgccgcctggaattggatcaggcaatctcctagtcggggtttggaatggttggggagaacttactaccgatccaagtggtacaacgactatgcagtttctgtcaagagtcgaatcacaatcaacccagatacttccaagaaccaattctctttgcagctgaatagtgtaacacccgaagataccgccgtttattattgcgcccgcgcctggggccctctctttgactattggggacagggcacattggtaactgtgtcctctggtggaggggggagtggagggggtggctcaggcggtagtgcgctttcctctgagctcacccaggaccccgccgtgagcgtagctttgggacagacagtccgaatcacatgtcagggcgattcactgagaagctattacgccagctggtaccagcagaaacctggacaggccccagttcttgttatctacggtaaaaataatagaccatctggtatccccgacaggttctctggctcctctagttcagggaacaccgcatcacttacaatcaccggcgcacaggctgaagatgaggctgactattactgcaactccagagactccagcggagcgatcgtgttcggtggtgggacgaaactgacggttttgggg

SEQ ID NO: 20 - protein, 246 aa, synthetic construct
MAQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARAWGPLFDYWGQGTLVTVSSGGGGSGGGGSGGSALSSELTQDPAVSVALGQTVRITCQGDSLRSYYASWYQQKPGQAPVLVIYGKNNRPSGIPDRFSGSSSSGNTASLTITGAQAEDEADYYCNSRDSSGAIVFGGGTKLTVLG

SEQ ID NO: 21 - DNA, 753 bp, synthetic construct
  source 1..753: mol_type = other DNA
gtcaacattagcggccagaacaccatgaacatggtaaaggtccctgaatgtcggttggctgatgaattgggcggactgtgggagaattccaggtttactgactgttgtctttgtgtggcagggcaagaattccaggcgcacaaggccattctcgctgctcggagcccagttttctcagccatgtttgagcacgagatggaggagtctaaaaaaaatcgggtagaaataaacgacgttgaaccagaagtctttaaagaaatgatgtgcttcatatataccggcaaggcaccaaatctcgacaaaatggcggacgatttgcttgctgcggctgataaatatgccctcgaacggttgaaggtgatgtgcgaggacgccctttgttctaatcttagcgtcgaaaacgcggctgaaatccttattctggcggaccttcattccgccgaccaactcaagacccaagccgtcgacttcataaactatcatgcttcagatgtgctggaaaccagcggctggaagagtatggtggtaagtcacccccatcttgttgcggaggcataccgaagtcttgcaagcgcgcaatgcccatttctgggaccaccacgcaagcgactcaagcaaagcgggagtggggcaaccaattttagtctgttgaaacaggccggagatgtggaagagaacccagggcctatggtatccaaaggagaggaagataacatggccataatcaaagagttcatgagattcaaggtccac

SEQ ID NO: 22 - protein, 251 aa, synthetic construct
VNISGQNTMNMVKVPECRLADELGGLWENSRFTDCCLCVAGQEFQAHKAILAARSPVFSAMFEHEMEESKKNRVEINDVEPEVFKEMMCFIYTGKAPNLDKMADDLLAAADKYALERLKVMCEDALCSNLSVENAAEILILADLHSADQLKTQAVDFINYHASDVLETSGWKSMVVSHPHLVAEAYRSLASAQCPFLGPPRKRLKQSGSGATNFSLLKQAGDVEENPGPMVSKGEEDNMAIIKEFMRFKVH

SEQ ID NO: 23 - DNA, 1950 bp, synthetic construct
  source 1..1950: mol_type = other DNA
cttgccagggctcatctccgggacgaggaaaaaagctgtccttgtttggcgcaggagggaccccaaggcgacctcttgacaaaaactcaggaactcggccgagattaccggacatgtctgaccattgtgcagaagttgaaaaagatggtcgataaaccgacacaaaggagcgtgagtaatgccgcaactagagtatgcagaacgggacgcagccgctggcgagacgtttgccgcaactttatgagacggtaccaaagtcgggtaacccagggtctcgtcgcgggcgaaacggcccaacagatatgtgaagatctgcgactctgcatccccagcacaggtcctcttggtggcggcggaagtgggggcggtggcagtgggggagggggttctatggctcaggtgcaactccagcaaagcggtccagggttggtaaagccatctcaaacgctgtctcttacttgtgctattagtggggattcagtttcaagcaattccgcagcttggaactggattagacaatcacctagccggggtttggagtggctcgggcgcacctattatcggtcaaagtggtataacgattacgcagtcagcgttaaaagccgcattacaataaacccggatacatccaagaaccagttttctctgcagctcaactctgtgacgcccgaggacactgccgtctattattgtgctagggcgtggggaccgctcttcgattactggggccagggaacattggttaccgtatcaagcgggggcgggggctcaggaggaggcggctctggagggtcagcacttagctctgagctgacgcaggatcctgctgtatcagttgcacttgggcagactgtacggataacctgccaaggggatagtctgcgctcttactacgcgtcatggtatcagcaaaaaccgggtcaagcacctgtcctggttatatacgggaagaataatagaccgtcaggtattcctgatcgattcagcggtagctccagtagcggtaacacagctagtctgactataacaggtgctcaagccgaggatgaagctgattattattgtaactcacgggacagcagtggggcgatagtcttcggcgggggcacgaagttgacggttctcggcggcggtggcggttctggtggaggcgggtctggaggcggaggttcagtaaacataagcggccaaaatactatgaacatggtgaaagtaccagaatgcaggttggcggatgagttggggggcctgtgggagaattcaagattcaccgactgctgtctgtgcgtcgctggacaggaattccaagcgcataaggcgatactggcggcacgaagtcctgtcttcagcgcgatgtttgaacatgagatggaagaatctaaaaaaaatagagtcgaaattaatgacgttgagccagaagtgttcaaagagatgatgtgctttatctatacaggaaaggcacctaaccttgataagatggcagatgacctgcttgctgctgctgataagtacgcgctggagcggttgaaagtcatgtgcgaggatgcattgtgttcaaacctttccgtcgagaacgccgcagaaatcctgattctcgccgatctgcacagcgctgatcagcttaagactcaggcagtagattttatcaactaccatgcctccgatgtgctcgagacatcaggatggaagtcaatggtagtttctcatccacatctcgtagctgaggcatatagatctctggcttcagcacaatgtccattccttggacccccaagaaagcgacttaagcaaagcgggtcaggagccaccaattttagcttgttgaagcaagcaggagacgtcgaggaaaatcctggtccgatggttagcaaaggtgaagaggacaatatggcgattatcaaagaattcatgagattcaaagtgcatgattataaagacgatgatgataag

SEQ ID NO: 24 - protein, 650 aa, synthetic construct
LARAHLRDEEKSCPCLAQEGPQGDLLTKTQELGRDYRTCLTIVQKLKKMVDKPTQRSVSNAATRVCRTGRSRWRDVCRNFMRRYQSRVTQGLVAGETAQQICEDLRLCIPSTGPLGGGGSGGGGSGGGGSMAQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARAWGPLFDYWGQGTLVTVSSGGGGSGGGGSGGSALSSELTQDPAVSVALGQTVRITCQGDSLRSYYASWYQQKPGQAPVLVIYGKNNRPSGIPDRFSGSSSSGNTASLTITGAQAEDEADYYCNSRDSSGAIVFGGGTKLTVLGGGGGSGGGGSGGGGSVNISGQNTMNMVKVPECRLADELGGLWENSRFTDCCLCVAGQEFQAHKAILAARSPVFSAMFEHEMEESKKNRVEINDVEPEVFKEMMCFIYTGKAPNLDKMADDLLAAADKYALERLKVMCEDALCSNLSVENAAEILILADLHSADQLKTQAVDFINYHASDVLETSGWKSMVVSHPHLVAEAYRSLASAQCPFLGPPRKRLKQSGSGATNFSLLKQAGDVEENPGPMVSKGEEDNMAIIKEFMRFKVHDYKDDDDK

(19) United States
(12) Patent Application Publication
(10) Pub. No.: US 2026/0125712 A1
(43) Pub. Date: May 7, 2026

(54) LYMPHOCYTE MEDIATED DELIVERY OF INTRACELLULAR TARGET-SPECIFIC PROTEINS

(71) Applicant: Saber Therapeutics, Inc., Coconut Creek, FL (US)

(72) Inventors: Aaron Welch, Bentonville, AR (US); Andrew Skora, San Clemente, CA (US); Christopher Bialas, Philadelphia, PA (US)

(73) Assignee: Saber Therapeutics, Inc., Coconut Creek, FL (US)

(21) Appl. No.: 19/217,761

(22) Filed: May 23, 2025

Related U.S. Application Data
(60) Provisional application No. 63/651,609, filed on May 24, 2024.

(51) Int. Cl. C12N 15/90; C12N 9/22; C12N 9/50; C12N 15/11

(52) U.S. Cl. CPC C12N 15/907; C12N 9/22; C12N 9/50; C12N 15/11; C12Y 301/21004; C12N 2310/20

(57) ABSTRACT

Provided herein are compositions of chimeric shuttle-binder-effector fusion proteins with components related to modulating endogenous cytotoxic effector mechanisms, which are useful for therapeutic effects on predetermined target molecules. These shuttle-binder-effector proteins also have specific epitope binding affinities for these target molecules. Compositions for modified cells expressing these chimeric shuttle-binder-effector fusion proteins are also provided. Methods for the modification of cells to express these chimeric shuttle-binder-effector fusion proteins and for delivering the chimeric shuttle-binder-effector fusion proteins into target cells using the lytic granule cellular mechanisms in these modified cells are also provided.

### CROSS-REFERENCE TO RELATED APPLICATIONS

This application claims priority to U.S. Provisional Application Ser. No. 63/651,609, filed May 24, 2024, the disclosure of which is incorporated herein by reference in its entirety.

### SEQUENCE LISTING

This application contains a Sequence Listing that has been submitted electronically as an XML file named “12429_012503-US1_SL_ST26.XML.” The XML file, created on May 23, 2025, is 39,847 bytes in size. The material in the XML file is hereby incorporated by reference in its entirety.

### TECHNICAL FIELD

This disclosure generally relates to the expression and delivery of a fusion protein capable of affecting specific target molecules in target cells.

### BACKGROUND

Cellular, or biologic, therapeutics for treatment of cancerous, or otherwise diseased, cells depend on membrane-bound markers or antigens present on the surface of target cells. Targeted therapeutics can bind to these markers via epitope-specific antibodies, or chimeric antigen receptors (CARs) on the surface of effector cells. However, for many cancers and other diseases, there are no known markers that are specific to the diseased cells.

Another method of treatment of cancerous, or otherwise diseased cells, is to use small molecules that are specific for a disease-causing protein. However, there are numerous proteins that are “undruggable” by this modality, having no easily discernable active or allosteric sites to inactivate. Furthermore, small molecules are limited by off-target toxicity caused by interaction with non-target molecules, or on-tumor off-target toxicity caused by interaction with target molecules in healthy tissues.

Several intracellular proteins are known to be drivers of disease but are difficult to affect by currently available methods. Hence, there is a significant clinical need to affect disease-driving proteins in diseased cells. The present described systems and methods address this need by using genetically modified lymphocytes designed to express a fusion protein, transferred into target cells via granzyme, that allows for perturbation of disease-causing proteins.

### SUMMARY

In one aspect, the subject matter of the foregoing application is directed to a chimeric molecule that includes fusion proteins and methods of delivering such fusion proteins into a target cell. For example, the one or more chimeric molecules described include a fusion protein that includes a shuttle domain, a binder domain, and an effector domain, with the domains being connected to one another by flexible linker sequences.

In a further arrangement, the shuttle domain of said fusion protein comprises all or part of a delivery protein, the binder domain of said fusion protein comprises all or part of a protein epitope binder, a sequence-dependent DNA-binding protein, or a sequence-dependent RNA-binding protein, and the effector domain of said fusion protein comprises a predetermined protein selected to have a predetermined effect on a target molecule. In a further implementation, the shuttle domain of the shuttle-binder-effector fusion protein includes a protein component of a lytic granule or a portion thereof. In another implementation, the binder domain of the fusion protein includes a sequence-dependent DNA-binding domain, wherein said sequence-dependent DNA-binding protein is a transcription activator-like effector (TALE) protein, or a zinc-finger (ZF) protein. In yet another implementation, the binder domain includes a protein epitope binder. Examples of this protein epitope binder include antibody mimetics such as designed ankyrin repeat proteins (DARPin), affibodies, and monobodies. Other examples include single-chain variable fragments (scFv), fragment antigen-binding regions (Fab) and heavy chain antibodies (HCab). In a further implementation, the protein epitope binder may cause inhibition or activation of the target molecule. In another aspect, the effector domain of the fusion protein can include a nuclease, kinase, ubiquitin ligase, or a phosphatase. In at least one aspect, the shuttle domain of the fusion protein includes a granzyme, the binder domain of the fusion protein includes a protein epitope binder, and the effector domain of the fusion protein includes a ubiquitin ligase or protease. In at least one other aspect, the shuttle domain of the fusion protein includes a granzyme, the binder domain of the fusion protein includes a sequence-specific DNA-binding domain or RNA-binding domain, and the effector domain of the fusion protein includes a nuclease.

One or more methods are directed to delivering a shuttle-binder-effector fusion protein into a target cell are also provided herein. In a particular arrangement, the fusion protein of the chimeric molecule delivered includes a shuttle domain, a binder domain, and an effector domain.

In a particular implementation, the subject matter of the foregoing application includes methods that describe genetically modifying a cell to create a modified cell that expresses a fusion protein. In yet a further implementation, the modified cell is a lymphocyte or a stem cell that can be activated to become a lymphocyte, creating one or more delivery cells from the modified cell, then delivering the shuttle-binder-effector protein into the target cells via contact with the delivery cells.

In a further aspect of the methods described herein, the shuttle domain of the shuttle-binder-effector fusion protein includes a component of a lytic granule. In one or more implementations, this component of a lytic granule includes a granzyme or a portion thereof, a granulysin or a portion thereof, a serglycin or a portion thereof, or a perforin or a portion thereof.

In a further aspect, the lymphocyte of the described method is further genetically modified to attenuate or eliminate expression of endogenous cytotoxic effector mechanisms. In an even further aspect, the endogenous cytotoxic effector mechanisms being attenuated or eliminated includes granzymes, Fas/FasL, and TRAIL pathways.

In a further aspect, the genetic modification of the lymphocyte cells is transient. In a further aspect, the genetic modification of the lymphocyte cells is stable. In a further aspect, contact between the delivery cells and the target cells is mediated via a chimeric-antigen receptor (CAR) that induces the formation of an immunologic synapse and secretion of the shuttle-binder-effector fusion protein. In a further aspect, the effector domain of the shuttle-binder-effector fusion protein comprises a nuclease, kinase, ubiquitin ligase, or a phosphatase.

In another aspect, the described systems and methods include a composition for delivery of a shuttle-binder-effector fusion protein to target cells, said composition comprising cytotoxic lymphocytes specific for the target cells, with those cytotoxic lymphocytes being genetically modified to express the shuttle-binder-effector fusion protein, wherein said fusion protein is sequestered in lytic granules of the cytotoxic lymphocytes, further wherein the shuttle-binder-effector fusion protein is deliverable into the target cell via the granzyme-perforin pathway when the cytotoxic lymphocytes interact with the target cells.

In a further aspect, the cytotoxic lymphocytes are primary lymphocytes, donor-derived lymphocytes, stem-cell derived lymphocytes, or cell-line derived NK cells, cell-line derived T cells, cell-line derived NKT cells, or cell-line derived macrophages. In a further aspect, the cytotoxic lymphocytes are modified to express the fusion protein stably. In a further aspect, the cytotoxic lymphocytes are modified to express the fusion protein transiently. In an even further aspect, the fusion protein comprises a granzyme, a protein epitope binder, and a ubiquitin ligase or protease. In an even further aspect, the fusion protein consists of a granzyme, a sequence-specific nucleic acid binder, and a nuclease.

### BRIEF DESCRIPTION OF THE FIGURES

FIG. 1A shows a diagram of an exemplary composition for engineered lymphocytes that have been modified to express a granzyme fusion protein.

FIG. 1B shows a diagram of an exemplary method for engineered lymphocytes delivering a granzyme fusion protein into target cells.

### DETAILED DESCRIPTION OF CERTAIN EMBODIMENTS

The present described systems and methods are directed to methods and compositions for delivering compounds, such as predetermined proteins, for example, shuttle-binder-effectors, to specific target cells. Aspects of the present described systems and methods are exemplified in a number of embodiments, some of which are summarized below and throughout the specification.

As used herein, “binding” or “binds” refers to an antibody-antigen mode or other highly specific mode of binding.

As used herein, “zinc finger proteins” refers to transcription factor proteins with the finger domain, which is an amino acid motif that recognizes to and binds to specific three-nucleotide sequences in DNA.

As used herein, “CRISPR system” refers to genetic engineering techniques involving the clustered regularly interspaced short palindromic repeats (CRISPR)-Cas nuclease system found in bacteria and archaea, as well as synthetic guide RNAs to create double-stranded breaks in genomic DNA and allow for permanent genome editing.

This application also makes reference to the following documents, which are herein incorporated by reference in their entirety: Baginska et al. PNAS. 2013 Oct. 22; 110(43):17450-5; Bekes M et al. Nature Reviews Drug Discovery. 2022 March; 21(3):181-200; Hlongwane et al. Biomedicines. 2018 Jun. 20; 6(2):72; PCT International Patent Application Publication No. WO 2015/157864, published Oct. 10, 2015 (Holt et al.); Hughes et al. Drug Discovery Today. 2021 Dec. 1; 26(12):2889-97; Lipoviek. Design & Selection. 2011 Jan. 1; 24(1-2):3-9; Miersch S, Sidhu S S. F1000Research. 2016; 5; Oberoi et al. PLoS One. 2013 Apr. 3; 8(4):e61267; Woodsworth et al. Molecular Therapy-Methods & Clinical Development. 2017 Dec. 15; 7:132-45; Lim et al. PNAS 2020 March 2; 117(11): 5791-5800; U.S. Patent Application Publication No. 2023/0024904, published Jan. 26, 2023 (Mashiach et al.); Restifo et al, Nature Reviews Immunology, 12: 269-281 (2011); Lupton S. D. et al., Mol. and Cell Biol., 11:6 (1991); Riddell et al., Human Gene Therapy 3:319-338 (1992); PCT International Application Publication No. WO 1992/008796, published May 29, 1992 (Lupton); PCT International Application Publication No. WO 1994/028143, published Dec. 8, 1994 (Lupton); Dudley et al, J. Immunotherapy, 26(4): 332-342 (2003); Dudley et al, Semin Oncol., 34(6): 524-531 (2007); U.S. Pat. No. 6,040,177, issued Mar. 21, 2000 (Riddell et al.); Kim et al, Nature Reviews Genetics, 15: 321-334 (2014); Gaj et al, Trends Biotechnology, 31(7): 397-405 (2013); Hsu et al, Cell, 157: 1262-1278 (2014); Sander et al, Nature Biotechnology, 32(4): 347-355 (2014); June et al, Nature Reviews Immunology, 9: 704-716 (2009); Schmidt et al, Biotechnology J., 10: 258-272 (2015); Mengstie, Front. Bioeng. Biotechnology, 12 May 2022:10:895713; Halene et al, Blood, 94: 3349-3357 (1999); Liechtenstein et al, Cancers, 5: 815-837 (2013); U.S. Pat. No. 9,678,061, issued Jun. 13, 2017 (Dornmair et al.); Breckpot et al., Gene Therapy, 14: 847-862 (2007); U.S. Patent Application Publication No. 2001/0007659, published Jul. 12, 2001 (Wong-Staal et al.); U.S. Pat. No. 8,951,535, issued Feb. 10, 2015 (Bauche and Sarry); Lupton S. D., et al, Mol. and Cell. Biology, 11:3374-3378 (1991); Coughlin et al, Blood, 103(6): 2046-2053 (2004); Dull et al, J. Virol., 72(11): 8463-8471 (1998); Naldini et al, Science, 272(5259): 263-267 (1996); PCT International Application Publication No. WO 2015/157864, published Oct. 22, 2015 (Holt et al.).; Prager and Watzl, J. Leukoc. Biol. 105: 1319-1329 (2019).

### Chimeric Fusion Proteins

In one or more embodiments, the subject matter described herein is directed to a chimeric fusion protein. In one or more embodiments, the fusion protein is a shuttle-binder-effector fusion protein. The domains of a shuttle-binder-effector fusion protein are connected to one another by flexible linker sequences, and the domains may be in any order. One example of a shuttle-binder-effector protein is a modified version of granzyme fused to a binder-effector protein, such as a protein that binds to an oncoprotein and causes it to be degraded, thereby removing the oncogenic impetus, and/or allowing the cell to apoptose. Examples of suitable linker sequences include G(4)S linkers and EAAAAK linkers. In one or more embodiments, the fusion proteins may be engineered to be delivered to selected target cells via the granzyme-perforin pathway. One such example of such a fusion protein is one that includes all or part of a delivery protein. In at least one particular embodiment, the fusion protein includes all or a portion of a component of a lytic granule. Examples of such components are a granzyme, a granulysin, or a serglycin. In one embodiment, the fusion protein may include a predetermined protein selected to be toxic to selected target cells; in another embodiment, the predetermined protein selected may be beneficial to the target cells.

In one or more embodiments, the binder domain of the fusion protein comprises a protein epitope binder. Some examples of suitable protein epitope binders include single-chain variable fragment (scFvs), antibody mimetics such as designed ankyrin repeat proteins (DARPins), affibodies, and monobodies, and heavy chain antibodies (HCab). Some examples of HCab include camelid VHH (nanobodies) and engineered variable heavy chains (VH). In at least one of those embodiments, the binder domain of the shuttle-binder-effector fusion protein includes a sequence-specific DNA-binding domain. Examples of such sequence-specific DNA-binding domains include zinc finger (ZF) proteins, transcription activator-like effector (TALE) proteins, homing meganucleases, or RNA-guided nucleases (RGNs). In at least one of those embodiments, an RGN is a component of a CRISPR system. In at least one other embodiment, the RGN is a Cas9 nuclease. In one or more of these embodiments, the delivery protein is a granzyme. In one or more of these embodiments, the predetermined protein is a toxin. In at least one embodiment, the predetermined protein can deliver the cytotoxic effect directly as a fusion protein. In another such embodiment, the fusion protein may undergo further processing before delivering its cytotoxic effect. One example of further processing is self-cleavage and release from a fusion protein prior to delivering a cytotoxic effect.

In one or more embodiments, the systems and methods described herein may include a fusion protein that does not include an entire delivery protein. In one or more embodiments, the fusion protein may include only a sequence motif from, or a portion of, a delivery protein which is necessary to sequester the protein in LGs and for transport to the cytosol of a target cell. In one or more embodiments, a delivery protein or portion thereof in a fusion protein may be only related to the natural delivery protein by a degree of amino acid homology. Some examples of the degree of homology the fusion protein can possess with the natural delivery protein are 90%, 80%, 70%, 60%, 50%, 40%, or 30% amino acid sequence identity with the delivery protein native to a delivery lymphocyte.

In one or more embodiments, only the C-terminal portion of a protein delivery protein may include a fusion protein. In one or more embodiments, the C-terminal portion may include less than 100%, 90%, 80%, 70%, 60%, 50%, 40%, 30%, 20%, 10%, or 5 percent of the amino acids of a delivery protein. In other embodiments, only the N-terminal portion of a protein delivery protein may include a fusion protein. In some embodiments, the N-terminal portion may include less than 100%, 90%, 80%, 70%, 60%, 50%, 40%, 30%, 20%, 10%, or 5% of the amino acids of a delivery protein. In one or more embodiments, an internal segment of a protein delivery protein may include a fusion protein. This internal segment may include less than 100%, 90%, 80%, 70%, 60%, 50%, 40%, 30%, 20%, 10%, or 5% of the amino acids of a delivery protein. The segment is also selected so that a fusion protein is sequestered in a lytic granule. In still further embodiments, such portion or segment is selected so that a fusion protein is sequestered in a lytic granule with at least fifty percent of the efficiency of the corresponding natural delivery protein.

### The Granzyme-Perforin Pathway

In one or more embodiments, the subject matter described herein is directed to methods of and systems for novel cell-based targeted delivery systems based on at least the granzyme-perforin pathway. In one or more embodiments, the described systems and methods provides cells having engineered granzyme-perforin pathways (hereafter “delivery cells”) for delivering to target cells predetermined compounds, preferably proteins, which may be designed to affect the target cells in a variety of ways. In one or more embodiments, the delivery cells can be engineered to deliver any of the fusion proteins described herein. The delivery cells may be engineered from any lymphocyte cell type that possesses, or can be engineered to possess, a granzyme-perforin pathway and a molecular recognition system for specifically binding to selected target cells. Although reference is made to a “granzyme-perforin pathway,” and equivalently to the “granzyme-perforin system” this is not meant to limit fusion proteins made in accordance with the described systems and methods to fusion of a granzyme and a predetermined or selected protein. In one or more embodiments, the cells being used to derive the delivery cells are lymphocytes. In one or more arrangements, the lymphocytes used to generate the delivery cells may be derived from stem cells, either induced-pluripotent stem cells or hematopoietic stem cells, or they may be primary lymphocytes isolated from donor sources. In at least one embodiment, the fusion protein is an epitope binder-E3-ubiquitin ligase-delivery protein fusion protein. Examples of suitable E3-ubiquitin ligases include von-hippel lindau (VHL), cereblon (CRBN), speckle-type pox virus and zinc finger protein (SPOP), and IpaH 9.8.

In one or more implementations of the systems and methods described, the delivery cells are engineered from cytotoxic lymphocytes. Cytotoxic lymphocytes are elements of the immune response that are mainly responsible for the recognition and clearance of cells infected by intracellular pathogens, as well as tumour immunosurveillance. Examples of cytotoxic lymphocytes include cytotoxic T lymphocytes (CTLs) and natural killer cells (NKs). Identification of target cells in cytotoxic lymphocytes is a complex process that hinges upon T cell receptor engagement of a cognate peptide presented by cell surface major histocompatibility complex (MHC) molecules. This interaction activates the key cytotoxic effector mechanisms of the cytotoxic lymphocytes, including the granzyme-perforin pathway. In humans there are at least five granzymes (granzyme A, granzyme B, granzyme H, granzyme K and granzyme M), of which granzyme B (GzB) is the best characterized.

GzB is a serine protease with a classical trypsin-like catalytic triad that initiates apoptosis in targeted cells. Primarily synthesized in cytotoxic lymphocytes as a precursor protein, GzB is directed to the endoplasmic reticulum by a signal peptide, which is cleaved to yield a zymogen form. The zymogen form of GzB is inactive due to an N-terminal dipeptide, but this dipeptide is cleaved by cathepsin C when GzB is localized to lytic granules (LGs), a type of specialized secretory lysosome. GzB is localized to LGs by post-translational modifications made in the Golgi network, which include mannose-6-phosphate and the chaperone molecule serglycin. Once GzB is localized to the LG and the dipeptide is cleaved, it is sequestered in the acidic LG and stored there awaiting CTL activation.

Granzyme A (GzA) is unique among the granzyme family in that it is the only member of the family that is found as a homodimer. It is expressed in many of the same cell types as GzA, but features different target residues for its protease activity (GzB is an Asp-ase, GzA is a tryptase). Granzyme K (GzK) has similar activity to GzA and is generally considered to be a “backup” enzyme for GzA, though it shows some unique function in that it can cleave the pre-mRNA-binding protein heterogenenous ribonuclear protein K. Granzyme H (GzH), which has chymase activity, cleaves adenovirus DNA-binding proteins and thus interferes with the viral replication process. Granzyme M (GzM) is a Met-ase that can cleave and inactivate Serpin B9 (SB9), which is an inhibitor of GzB. GzM is also expressed endogenously in mice and rats. GzA, GzB, GzH, and GzK follow similar synthesis and processing pathways to the one described for GzB, with the partial exception of GzA, where two mRNAs arise from alternative splicing of different exons 1. GzB has the strongest apoptotic function of the granzyme family, but all members of the family are involved in the cell death pathway. GzA has also been theorized be involved in regulating B-cell proliferation.

The other major component of this pathway is perforin, a protein that forms pores in targeted cells and is stored in LGs alongside granzyme-serglycin aggregates. Upon T cell receptor (TCR) engagement in an activated CTL, a tight enclosed region between the CTL and the target cell is formed. This region is known as the immunological synapse (IS). Perforin and granzyme are exocytosed from the LGs into the IS and diffuse across to the membrane of the target cell. Once inside the target cell, perforin aggregates to form multimeric transmembrane pores. The pores seem to be only briefly present before membrane integrity is restored, with their main function being a conduit for passive diffusion of delivery proteins (such as granzyme B and the other granzymes and granulysin which are co-packaged with GzB in LGs) into the target cell. Once in the cytosol, the delivery proteins in the LGs initiate apoptosis. GzB cleaves BH3 interacting-domain death agonist (BID) and caspases 3, 7 and 8, which in turn activate the mitochondrial and caspase apoptosis pathways, respectively. In summary, the synergistic activities of granzyme and perforin represent a unique pathway for transferring molecules from CTLs to target cell exclusively, as the immunological synapse confines granzyme and perforin between the two cells, and moreover, significant numbers of perforin molecules are required to form the pores required for granzyme's entry into the target cell.

In one or more embodiments, the described systems and methods include the use of genetically modifying cytotoxic lymphocytes. Some example genetic modifications of cytotoxic lymphocytes include, (i) the addition of genes for targeting capability, e.g. genes that encode chimeric antigen receptors (CARs), or similar targeting molecules; (ii) the addition of genes that encode one or more fusion proteins, each such gene including a delivery protein or a portion thereof and any of the fusion proteins described herein; (iii) the deletion of apoptosis-inducing receptors, such as FasR or TRAIL in NK cells; and (iv) other genetic modifications to enhance lymphocyte potency, proliferation, or persistence.

In one or more embodiments, the granzyme-perforin pathway comprises (i) lytic granules containing one or more delivery proteins including perforin, and (ii) an activatable intracellular transport system for moving lytic granules to the delivery cell surface membrane for exocytosis and release of the one or more delivery proteins, such that released perforins form pores in the surface membrane of a recognized target cell. Under these conditions, released non-perforin delivery proteins, including fusion proteins, are transported by diffusion into the cytosol of the target cells. The activatable intracellular transport system is activated directly or indirectly by specific recognition of a target cell by the delivery cell. Recognition may be accomplished by specific binding of targeting molecules on the surface of the delivery cells to one or more antigens on the surface of the target cells. For example, recognition may be accomplished by specific binding of a TCR on a delivery cell, such as a CTL, to an MHC-epitope complex on the surface of a target cell; or recognition may be accomplished by ligation of activating receptors on a delivery cell, such as an NK cell; or recognition may be accomplished by specific binding of a CAR on a delivery cell to a predetermined antigen of the surface of a target cell. Other molecular or metabolic targeting structures known to those with skill in the art may also be employed with granzyme-perforin pathways using conventional cellular engineering methods.

In one aspect, the described systems and methods provide a novel approach to therapeutic molecule delivery by modifying the granzyme-perforin pathway of cytotoxic lymphocytes. In some embodiments, this modification comprises expressing a delivery protein fused to a peptide epitope binder fused to an E3-ubiquitin ligase, which is sequestered in lytic granules followed by delivery to target cells. In some embodiments, such engineering comprises expressing a delivery protein fused to a protein that binds a specific sequence of RNA fused to a nuclease. Some examples of these protein epitope binders include the fusion proteins described herein. When combined with antigen receptor-mediated targeting, the described systems and methods provide a new class of cell-based therapeutics with improved efficacy and lower iatrogenic toxicity. The invention is complementary with other cell therapies, including hematopoietic stem cell transplants (HSCTs) and adoptive transfer of tumor reactive lymphocytes, such as Chimeric Antigen Receptor (CAR)-engineered lymphocytes. In some embodiments, methods of the invention include antigen receptor mediated targeting and a safety suicide system. In some embodiments, the method enables the cytoplasmic delivery of predetermined protein payloads in a highly specific manner to a target cell population.

Upon activation of a cytotoxic lymphocyte, delivery proteins such as perforin and granzyme are released from the cytoplasmic LGs in which they are stored, into the immunological synapse that forms between a cytotoxic lymphocyte and its target cell. Perforin inserts into the target cell membrane and aggregates to form multimeric, transmembrane pores. Perforin alone is not cytotoxic at physiological concentrations, rather it functions to permit passive but highly localized diffusion of granzyme into the target cell. Once in the cytosol of the target cell, the granzyme, primarily GzB in humans, initiates apoptosis by cleavage of BID and caspases 3, 7 and 8. In one or more embodiments, the described systems and methods provide a method of using this pathway for the purpose of delivering a protein, such as a shuttle-binder-effector, by using GzB, or another lytic granule protein, as a molecular chaperone to carry a shuttle-binder-effector fusion protein into a target cell, such as a tumor cell. In one embodiment, this is achieved by fusing a shuttle-binder-effector fusion protein to the intact GzB protein such that upon transfection of a lymphocyte with a vector encoding the fusion protein, it will be expressed and packaged into lytic granules and released upon specific binding of the cytotoxic lymphocyte to target cells. Some examples of this binding interaction include via T cell receptor binding to a MHC-peptide complex, and a CAR binding to a target epitope.

These and other aspects of the present methods and compositions are described in further detail below with reference to the accompanied drawing figures, in which one or more illustrated embodiments and/or arrangements of the methods and compositions are shown. In at least one other embodiment, a similar effect can be achieved by fusing a shuttle-binder-effector fusion protein with another granzyme protein, such as GzA, GzH, GzK, or GzM.

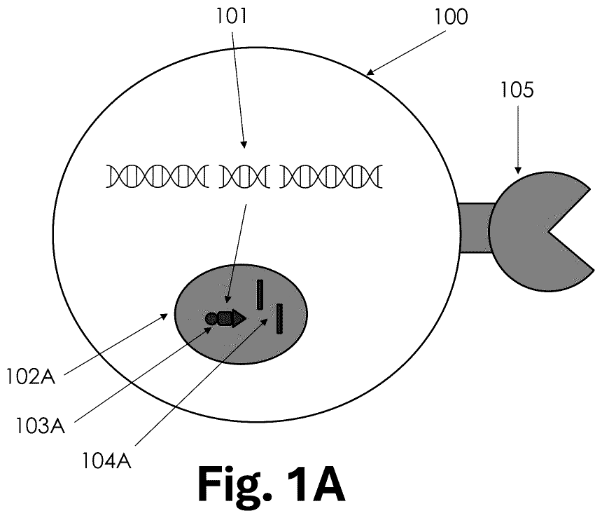

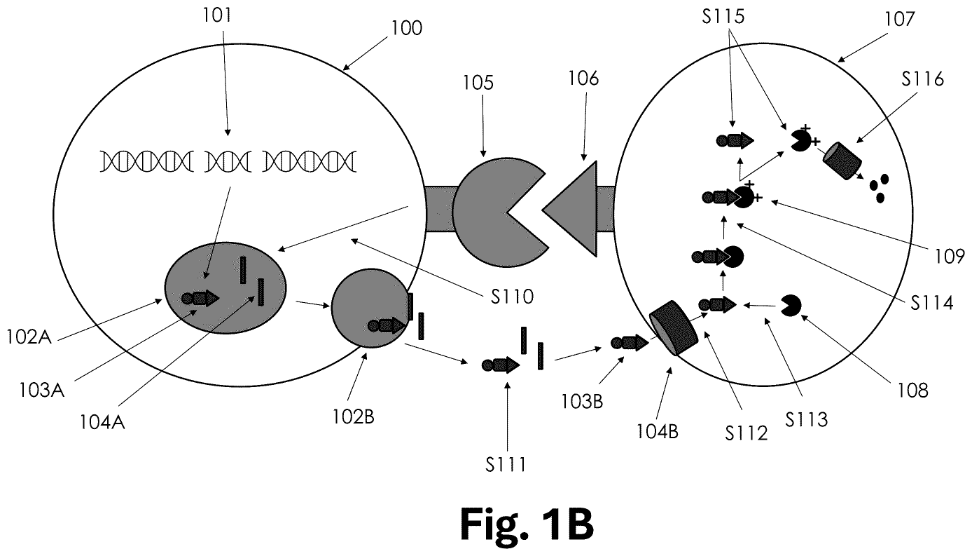

FIG. 1A shows a diagram of an exemplary composition for engineered lymphocytes 100 to be used in accordance with one or more of the embodiments of the described systems and methods. With reference now to FIG. 1A, in one or more embodiments, the engineered lymphocyte 100 has been modified with a stably integrated transgene 101. This stably integrated transgene 101 has been integrated with a section of the genome such that the lytic granule 102A contains a copy of a granzyme fusion protein 103A along with perforin 104A. In accordance with one or more embodiments, the engineered lymphocyte identifies its target cells using a target cell identifier 105. In some embodiments, this identifier can be a CAR. In other embodiments, this identifier can be a T cell receptor.

FIG. 1B shows a diagram of an exemplary method for using engineered lymphocytes 100 with a stably integrated transgene 101 as a delivery system for lytic granules 102A containing a granzyme fusion protein 103A and perforin 104A in accordance with one or more embodiments of the present systems and methods. With reference now to FIG. 1B, in one or more embodiments, the engineered lymphocyte 100 induces transport of activated lytic granules 102B to the cell surface as part of step S110. Once the lytic granules 102B are on the cell surface, they will secrete their cargo into the immune synapse upon ligation with the cognate antigen 106 on the target cell 107 as part of step S111. Once the engineered lymphocyte 100 is ligated with the target cell 107, perforin 104B mediates the delivery of the granzyme fusion protein 103B to the cytosol of the target cell 107 as part of step S112. Once the granzyme fusion protein 103B is in the cytosol of the target cell 107, the protein binder domain of the granzyme fusion protein 103B binds to the target molecule 108 as part of step S113. In at least one embodiment, the target molecule 108 is an oncoprotein, and the granzyme fusion protein 103B contains an E3 ubiquitin ligase, which causes ubiquitination of the oncoprotein, as described in step S114. Once the oncoprotein has been ubiquitinated, the ubiquitinated oncoprotein and the granzyme fusion protein 103B separate, as described in step S115. The ubiquitinated oncoprotein 109 is subsequently degraded by the proteasome, as described in step S116.

In some embodiments, this lymphocyte-based delivery system may be used to deliver binder-effector proteins, such as those described herein. In at least one embodiment, the delivery cells for these binder-effector proteins are produced and administered using adoptive cell therapy (ACT) techniques, such as those described in Restifo et al, Nature Reviews Immunology, 12: 269-281 (2011), which is herein incorporated by reference. This lymphocyte-mediated delivery of binder-effector proteins provides a new modality for cancer treatment.

Refractory cancer often results from loss of response to apoptotic signals, primarily via down regulation of executioner caspases 3 and 7 and/or up-regulation of inhibitor of apoptosis proteins (IAPs). Such forms of resistance have been observed in many malignancies and correlate significantly with poor survival and other negative outcomes, which underscores the need for new therapies. The ability of tumors to evade immune responses remains a significant challenge for cancer therapy. CTLs and NK cells exert their natural cytotoxic effects primarily by delivering delivery proteins, such as granzymes, via perforin pores to target cells, where, for example, granzymes subsequently cleave target cell caspases to induce target cell apoptosis. However, as mentioned above, tumor cells are often apoptosis resistant, or acquire such resistance. In fact, apoptosis evasion is a hallmark of cancer and chemotherapy resistance. In or more embodiments, cytotoxic lymphocytes are modified by adding a gene that encodes a fusion protein, such as one of the fusion proteins described herein. In some embodiments, the described systems and methods include a method of cancer treatment, this method further comprising modifying cytotoxic lymphocytes derived from a patient with the gene for the shuttle-binder-effector protein combination, then re-administering these modified lymphocytes to the patient. In at least one of these embodiments, the described systems and methods include a method treating a patient with cancer, this method further comprising (a) genetically modifying cytotoxic lymphocytes so that they have the capability of expressing at least one shuttle-binder-effector protein in lytic granules, (b) expanding the genetically modified cells, and (c) administering the genetically modified cells to the patient. In at least one of these embodiments, the method further includes obtaining cytotoxic lymphocytes from white blood cells of the patient or of MHC-matched donors. In at least one embodiment, the cytotoxic lymphocytes may be derived from cell lines or engineered cells. One example of such a cell line is the NK-92 cell line. In one or more embodiments, the cytotoxic lymphocytes may be further genetically modified to express a CAR to direct the cytotoxic lymphocytes to specific target cells. In at least one embodiment, these cytotoxic lymphocytes may be further modified to enhance persistence, trafficking, targeting, efficacy, or safety.

All current cell therapies for cancer that are being developed and tested rely on the natural cytotoxicity of lymphocytes. That is, the natural degranulation of the lymphocyte against its target tumor cell and granzyme mediated induction of target cell apoptosis. Similarly, most current targeted monoclonal antibody therapeutics for cancer rely, ultimately, on endogenous cytotoxic lymphocytes (either via antibody dependent cellular cytotoxicity, ADCC, or by disinhibiting lymphocytes via checkpoint receptor blockade) for their tumoricidal effects. In accordance with some embodiments of the present systems and methods, by exploiting delivery proteins of cytotoxic T cells or NK cells as delivery vehicles for a shuttle-binder-effector molecule, apoptosis resistance can be circumvented.

In some embodiments with therapeutic applications, (a) a biological cell is used as a delivery vehicle, which has substantial advantages, including (i) protecting the delivered molecule from immunological clearance as well as constitutive breakdown; (ii) enabling antigen mediated selection of targeted tissue; (iii) achieving widespread tissue distribution, (iv) a granzyme-perforin system inherent in these lymphocytes is used to enable extremely specific cell-to-cell delivery of an arbitrary predetermined protein, while all but eliminating off-target effects; and (v) a cell-based delivery system offers the opportunity to add additional functions such as sensing the tumor microenvironment via hypoxia sensors, or including suicide switches to improve in vivo control and safety.

The specificity of delivery to desired target cells relies on the specificity of the endogenous antigen receptor on the engineered, or carrier, lymphocyte, or alternatively, in some embodiments, the specificity of a CAR used to co-modify the engineered lymphocyte, or an additional sensing module used to co-modify the engineered lymphocyte.

### Sources of Cytotoxic Lymphocytes

In one or more embodiments, cytotoxic lymphocytes are used in compositions of matter and methods of the disclosed systems and methods. The cells may be obtained from immortalized cell lines, such as immortalized NK cell lines. Some examples of immortalized cell lines are NK92, HL-60, and YT. Alternatively, they may be obtained from patients or donors (i.e. primary cells) by a variety of techniques known in the art, particularly the art of adoptive cell therapy. Examples of this technique described in Dudley et al, J. Immunotherapy, 26(4): 332-342 (2003) and Dudley et al, Semin Oncol., 34(6): 524-531 (2007), both of which are hereby incorporated by reference as if presented in their entirety. Additionally, both NK and T cells may be derived from induced pluripotent stem cells (iPSCs).

T lymphocytes can be collected in accordance with techniques known in the art, including enrichment and depletion techniques. Examples of these techniques include discontinuous density gradient centrifugation, magnetic bead affinity separation, and fluorescently activated cell sorting (FACS). Both alpha-beta and gamma-delta T cells could be used. After the enrichment and/or depletion steps, in vitro expansion of the desired T lymphocytes can be carried out in accordance with known techniques. Examples of these techniques include those described in U.S. Pat. No. 6,040,177 to Riddell et al., which is hereby incorporated by reference, or variations thereof that will be apparent to those skilled in the art.

For example, the desired T cell population or subpopulation may be expanded by adding an initial T lymphocyte population to a culture medium in vitro, and then adding to the culture medium feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). The order of addition of the T cells and feeder cells to the culture media can be reversed if desired. The culture can typically be incubated under conditions of temperature and the like that are suitable for the growth of T lymphocytes. For the growth of human T lymphocytes, for example, the temperature will generally be at least about 25 degrees Celsius, preferably at least about 30 degrees, more preferably about 37 degrees. The T lymphocytes expanded are typically cytotoxic T lymphocytes (CTL) that are specific for an antigen present on a human tumor or a pathogen. The non-dividing feeder cells can comprise gamma-irradiated PBMC feeder cells. In some embodiments, the PBMC are irradiated with gamma rays in the range of about 3000 to 3600 rads. Optionally, the expansion method may further comprise the step of adding non-dividing EBV-transformed lymphoblastoid cells (LCL) as feeder cells. LCL can be irradiated with gamma rays in the range of about 6000 to 10,000 rads. The LCL feeder cells may be provided in any suitable amount, such as a ratio of LCL feeder cells to initial T lymphocytes of at least about 10:1. Optionally, the expansion method may further comprise the step of adding anti-CD3 monoclonal antibody to the culture medium (e.g., at a concentration of at least about 0.5 ng/ml). Optionally, the expansion method may further comprise the step of adding IL-2 and/or IL-15 to the culture medium (e.g., wherein the concentration of IL-2 is at least about 10 units/ml).

In some embodiments, it may be desirable to introduce functional genes into the T or NK cells to be used in immunotherapy. One example of desirable functional genes are genes that improve the efficacy of the invention by promoting the viability and/or function of transferred T cells. Other examples are genes that provide a genetic marker to permit selection and/or evaluation of in vivo survival or migration or genes that incorporate functions that improve the safety of immunotherapy, such as making the cell susceptible to negative selection in vivo as described by Lupton S. D. et al., Mol. and Cell Biol., 11:6 (1991); and Riddell et al., Human Gene Therapy 3:319-338 (1992), both of which are hereby incorporated by reference.¹

In one or more embodiments, the delivery cells are engineered to couple a cellular recognition system (such as, T cell receptors, CARs, or the like) with granzyme-perforin systems ¹See also PCT International Application Publication Nos. WO 1992/008796 (Lupton) and WO 1994/028143 (Lupton), which describe the use of bifunctional selectable fusion genes derived from fusing a dominant positive selectable marker with a negative selectable marker. This can be carried out in accordance with techniques known in the art, such as what is described in U.S. Pat. No. 6,040,177 (Riddell et al.) at columns 14-17.

Engineering Lymphocytes to provide cell-based compound delivery to targeted cell populations. In one or more embodiments, these delivery cells are lymphocytes that already possess one or both of these capabilities, which may be modified using conventional genetic engineering techniques to permit delivery of predetermined compounds, such as shuttle-binder-effector proteins. In one or more embodiments, cytotoxic lymphocytes are genetically modified so that they produce a fusion protein comprising a shuttle-binder-effector protein which is sequestered in a lytic granule. In at least one such embodiment, such fusion proteins sequestered in lytic granules are released in response to specific recognition of an MHC-epitope complex on a target cell by a receptor of the engineered cytotoxic lymphocyte. In at least one other embodiment, innate activating receptors may cause release of lytic granules. In at least one other embodiment, specific recognition of a target cell may be accomplished by cytotoxic lymphocytes expressing a CAR specific for a surface antigen of the target cell.

Some example techniques that can be used to carry out the genetic modifications discussed above are conventional mammalian cell genetic engineering techniques such plasmid or RNA transfection, transduction by viral vectors and direct genome editing using programmable nucleases, such as CRISPR systems, TALE proteins, and ZF proteins. Guidance for applying such techniques to the presently described systems and methods may be found in the following references, which are incorporated herein by reference as if presented in their respective entireties: Kim et al, Nature Reviews Genetics, 15: 321-334 (2014); Gaj et al, Trends Biotechnology, 31(7): 397-405 (2013); Hsu et al, Cell, 157: 1262-1278 (2014); Sander et al, Nature Biotechnology, 32(4): 347-355 (2014); June et al, Nature Reviews Immunology, 9: 704-716 (2009); Schmidt et al, Biotechnology J., 10: 258-272 (2015); Senis et al, Biotechnology Journal, 9: 1402-1412 (2014) (including supplemental materials).

In one or more embodiments, genetic modification is made by transducing cytotoxic lymphocytes using a vector that stably integrates into its genome. Exemplary vectors for such transduction include lentivirus and retrovirus vectors, adenoviruses, adeno-associated virus (AAV), and transposons. In at least one of these embodiments, retroviral vectors are used to transduce cytotoxic lymphocytes, as is taught by Halene et al, Blood, 94: 3349-3357 (1999), which is incorporated by reference. In one or more embodiments, a lentivirus vector is employed to transduce cytotoxic lymphocytes. Lentivirus vectors that can readily be modified to incorporate fusion proteins are commercially available. Examples of modifying and using lentivirus vectors is provided in the following references, which are incorporated herein by reference as if presented in their respective entireties: Liechtenstein et al, Cancers, 5: 815-837 (2013); U.S. Pat. No. 9,678,061 (Dornmair et al.); Breckpot et al., Gene Therapy, 14: 847-862 (2007); U.S. Patent Application Publication No. 2001/0007659 (Wong-Staal et al.); and U.S. Pat. No. 8,951,535 (Bauche and Sarry).

In one or more embodiments, a methods of treating an individual with an apoptotic resistant disease, such as a cancer, comprises obtaining cytotoxic lymphocytes from an individual, genetically modifying the isolated cytotoxic lymphocytes so that they are capable of producing lytic granules that sequester a fusion protein comprising a shuttle-binder-effector protein, expanding the genetically modified cytotoxic lymphocytes to generate therapeutic amounts of cells, and administering to the individual a therapeutically effective amount of the genetically modified cytotoxic lymphocytes. In at least one of those embodiments, the cytotoxic lymphocytes are genetically modified by transducing the cytotoxic lymphocytes using a viral vector.

In one or more embodiments, a positive marker may be included in the delivery cells. This positive marker enables the selection of cells of the negative selectable phenotype in vitro. The positive selectable marker may be a gene which, upon being introduced into the host cell, expresses a dominant phenotype permitting positive selection of cells carrying the gene. Some examples of these gene include hygromycin-B phosphotransferase gene (hph), which confers resistance to hygromycin B; the aminoglycoside phosphotransferase gene (neo or aph) from Tn5, which codes for resistance to the antibiotic G418; the dihydrofolate reductase (DHFR) gene; the adenosine deaminase gene (ADA); and the multi-drug resistance (MDR) gene. These positive markers, also known as resistance markers, are useful when transfecting a plasmid into cytotoxic lymphocytes. Plasmids are episomal and do not replicate; thus, they are eventually lost or diluted by repeated cell division. The use of a selective marker enables selection of cells where the plasmid has been integrated into the genome.

In the embodiments where viral transduction is used, genome integration is routine, and selection using a resistance marker is therefore unnecessary. Instead, a fluorescent protein driven by an appropriate mammalian promoter may be included in the virus. When the virus integrates into the genome, the fluorescent protein will be expressed as a marker to sort or otherwise follow the transduced cell population. A classic example of such a marker is a gene for a fluorescent protein, such as green fluorescent protein (GFP).

In one or more embodiments, the positive selectable marker and the negative selectable element are linked such that loss of the negative selectable element necessarily also is accompanied by loss of the positive selectable marker. In at least one of those embodiments, the positive and negative selectable markers are fused so that loss of one obligatorily leads to loss of the other. An example of a fused polynucleotide that yields as an expression product a polypeptide that confers both the desired positive and negative selection features described above is a hygromycin phosphotransferase thymidine kinase fusion gene (HyTK). Expression of this gene yields a polypeptide that confers hygromycin B resistance for positive selection in vitro, and ganciclovir sensitivity for negative selection in vivo. See Lupton S. D., et al, Mol. and Cell. Biology, 11:3374-3378 (1991), which has been incorporated by reference. In at least one embodiment, the polynucleotides of the described systems and methods encoding the chimeric receptors are in retroviral vectors containing the fused gene, particularly those that confer hygromycin B resistance for positive selection in vitro, and ganciclovir sensitivity for negative selection in vivo. One example is the HyTK retroviral vector described in Lupton, S. D. et al. (1991), supra. See also PCT International Application Publication No. WO 1992/008796 (Lupton) and PCT International Application Publication No. WO 1994/028143 (Lupton).

Some examples of positive selectable markers that can be used in one or more embodiments of the described systems and methods are derived from genes selected from the group consisting of hph, neo, and gpt, and preferred negative selectable markers are derived from genes selected from the group consisting of cytosine deaminase, HSV-I TK, VZV TK, HPRT, APRT and gpt. In at least one of these embodiments, the markers are bifunctional selectable fusion genes wherein the positive selectable marker is derived from hph or neo, and the negative selectable marker is derived from cytosine deaminase or a TK gene. In at least one embodiment, negative selectable markers are also used in the final product to be given to humans. These serve to eliminate the modified cytotoxic lymphocytes in the event of safety issues arising from the cytotoxic lymphocytes.

A variety of methods can be employed for transducing lymphocytes in accordance with one or more embodiments of the described systems and methods. An example protocol for transducing lymphocytes is to provide the cells with 20-30 units/ml IL-2 after stimulation using REM on day 1(as described in U.S. Pat. No. 6,040,177); then replace one half of the medium with retroviral supernatant prepared according to standard methods and then supplementing the cultures with 5 μg/ml polybrene and 20-30 units/ml IL-2 on day 3; then wash the cells and place them in fresh culture medium supplemented with 20-30 units/ml IL-2 on day 4; then repeat the retroviral exposure on day 5; then place the cells in a selective medium (e.g. medium containing an antibiotic corresponding to an antiobiotic resistance gene provided in the retroviral vector) supplemented with 30 units/ml IL-2 on day 6; and then separate viable cells from dead cells using Ficoll Hypaque density gradient separation and subclone the viable cells on day 13.

In one or more embodiments, transgenes may be introduced for expression in cytotoxic lymphocytes by in vitro RNA transfection, for example, as disclosed by Coughlin et al, Blood, 103(6): 2046-2053 (2004), which is incorporated herein by reference. The protocol described by this reference is that RNA capable of expressing a fusion protein of interest is transcribed in vitro, then electroporated into the desired cells. After uptake, the target cells express the RNA to produce the fusion protein of interest.

In one or more embodiments, lentiviruses may be used to insert fusion proteins and other transgenes into the delivery cells. Lentiviral vectors can mediate the efficient delivery, integration and stable or controlled expression of transgenes in dividing as well as non-dividing cells in vitro. Kits and materials for constructing lentivirus vectors are available commercially, and guidance for their application is found in the following exemplary references, which are hereby incorporated by reference: Dull et al, J. Virol., 72(11): 8463-8471 (1998); Naldini et al, Science, 272(5259): 263-267 (1996). Briefly, a common protocol calls for construction of at least three precursor vectors: a transfer vector containing a transgene, an envelope vector and a packaging vector. The three vectors are co-transfected into a virus production cell line, such as A293T cells, which serve as a production host for packaged lentivirus. The packaged lentivirus is harvested from the A293T culture and adjusted to a titer for infecting target cytotoxic lymphocytes.

In one or more embodiments, cytotoxic lymphocytes may be engineered using a CRISPR system to express fusion proteins for delivery target cells. With this approach, a fusion protein may be created by directly modifying the granzyme genomic locus in NK or T cells. Some examples of suitable genetic modification systems include a CRISPR system, or a like system (such as ZF proteins, or TALE proteins) for direct genomic modification. In some embodiments, such modification may be carried out in vitro or in vivo, where the transgene (for example, encoding a fusion protein) and cas9 gene and guide RNAs are delivered by a viral vector like AAV, LV, or ADV, such as the ones described in Mengstie, Front. Bioeng. Biotechnology, 12 May 2022:10:895713. This approach is advantageous because the transgene would be under the control of the native granzyme promoter and other native regulatory elements, which may be important for optimal expression of the fusion protein and loading into lytic granules. It can also reduce possible inefficiencies due to competition by the native granzyme for vesicle loading with granzyme fusion proteins from transfection or transduction.

In one or more embodiments, cytotoxic lymphocytes may be genetically modified by the following steps: (i) transfecting cytotoxic lymphocytes for constitutive or transient expression transgenes encoding a programmable nuclease; (ii) transfecting the cytotoxic lymphocytes with donor templates for homology-directed repair of double stranded breaks produced by the template-specific nuclease, the donor templates encoding a fusion protein including a shuttle-binder-effector protein, such as any of the ones described here in, and also including sequences to target the donor template to the double stranded break produced by the template-specific nuclease. In one or more of these embodiments, the donor template is a component of a double stranded plasmid. In one or more embodiments, the donor template is a component of a viral vector. In at least one of these embodiments, the step of transfecting cytotoxic lymphocytes with an RGN and guide RNA results in transient expression thereof. In at least one of these embodiments, the step of transfecting cytotoxic lymphocytes with an RGN and guide RNA is carried out with a viral vector. Some examples of a suitable viral vector include lentiviruses, AAVs, gammaretroviruses, spumaviruses, Ad5-35, and lymphotropic herpesviruses.

Although much of the foregoing description has been directed to lymphocyte mediated delivery of intracellular target-specific proteins, the methods and compositions disclosed herein can be similarly deployed and/or implemented in scenarios, situations, and settings far beyond the referenced scenarios. It should be further understood that any such implementation and/or deployment is within the scope of the methods described herein.

### EXAMPLES

The invention will be described in greater detail by way of specific examples. The following examples are offered for illustrative purposes and are not intended to limit the invention in any manner. Persons of skill in the art will recognize the presence of non-critical parameters which can be changed or modified to yield similar results.

### Example 1: Targeted Cancer Therapy Using CAR-T Cells

This example will describe a method for treating CD19-positive cancers using CAR-T cells engineered to express a fusion protein. The fusion protein, comprising a Granzyme B shuttle domain, an scFv binder targeting the intracellular oncoprotein BCL2, and the E3 ubiquitin ligase effector Cereblon, enhances therapy by degrading the oncoprotein inside cancer cells, leading to apoptosis.

### Construct Design and Cloning Strategy

A CD 19-specific CAR will be designed. This CAR will have an scFv domain, a IgG1 hinge domain, a transmembrane domain, 4-1BB costimulatory domain, and CD3ζ signaling motif. A fusion protein (FP1) gene will be designed with a truncated Granzyme B shuttle (amino acids 21-247), an scFv binder for BCL2, and a modified cereblon ligase, linked by a (Gy4Ser)3 linker, with an optional FLAG tag. The sequences for these constructs are described in Table 1, below.

TABLE 1
Sequences of CD19-Specific CAR and FP1 Components

| CAR or FP1 Component | Sequence |
|---|---|
| CD-19 specific CAR, nucleic | AATGCTCCTTTTGGTCACATCTTTGTTGCTTTGCGAACTGCCTCAT |
| acid (SEQ ID NO: 1) | CCAGCGTTCTTGCTTATACCAGATATCCAGATGACACAGACGACC |
| | TCCAGCCTCTCAGCTTCCTTGGGGGATAGGGTAACCATTTCTTGT |
| | AGAGCTTCCCAAGATATCAGCAAGTACCTTAATTGGTATCAGCAG |
| | AAGCCCGATGGAACTGTGAAGTTGCTGATTTACCACACATCTAGA |
| | TTGCACTCCGGAGTACCGAGCCGCTTCAGCGGCTCAGGATCAGG |
| | GACTGACTACTCACTGACTATCAGCAATCTTGAACAAGAGGACAT |
| | TGCGACTTATTTTTGCCAACAGGGAAACACACTGCCTTATACCTT |
| | TGGAGGCGGTACAAAACTTGAAATAACAGGATCTACCTCTGGATC |
| | CGGTAAACCAGGGTCCGGAGAAGGATCTACCAAAGGTGAAGTCA |
| | AGCTCCAAGAAAGCGGACCAGGCCTTGTTGCACCCAGCCAATCT |
| | TTGTCAGTTACGTGCACAGTGTCAGGCGTATCCTTGCCGGACTAC |
| | GGCGTGTCATGGATCAGACAACCCCCTAGAAAAGGTCTGGAGTG |
| | GCTGGGAGTGATATGGGGAAGTGAAACTACCTATTATAATAGTGC |
| | GCTTAAATCACGGCTCACAATTATAAAGGACAATTCTAAGTCACA |
| | AGTCTTCTTGAAGATGAACTCTCTTCAAACGGACGACACCGCCAT |
| | TTACTACTGCGCTAAGCATTACTATTATGGGGGGTCCTATGCAATG |
| | GATTATTGGGGCCAAGGGACCTCTGTAACGGTGAGTAGCGCAGC |
| | TGCCACAACGACCCCGGCACCTCGACCACCAACCCCAGCGCCTA |
| | CAATTGCGTCCCAACCCTTGAGCCTTAGGCCAGAGGCATGCCGG |
| | CCCGCGGCTGGCGGAGCTGTGCATACTCGCGGATTGGATTTTGCT |
| | TGTGACATCTACATTTGGGCCCCACTCGCTGGGACCTGTGGGGTC |
| | TTGCTTCTGTCCTTGGTAATAACTTTGTACTGCAAGCGGGGGCGG |
| | AAAAAACTCCTCTATATTTTCAAACAACCTTTTATGAGACCAGTTC |
| | AGACAACGCAGGAAGAGGATGGCTGCTCATGTCGGTTTCCCGAG |
| | GAGGAAGAAGGTGGTTGTGAACTGCGAGTTAAGTTCTCCAGATC |
| | AGCAGATGCCCCGGCATATCAGCAGGGGCAAAACCAACTCTATA |
| | ATGAGTTGAATCTCGGCAGAAGGGAGGAATATGATGTGCTGGAC |
| | AAAAGAAGAGGCAGGGACCCGGAGATGGGAGGGAAACCTAGGC |
| | GAAAAAACCCTCAAGAGGGACTTTATAACGAACTTCAGAAAGAC |
| | AAGATGGCAGAGGCGTACAGTGAAATTGGTATGAAAGGTGAAAG |
| | ACGAAGGGGGAAGGGACACGATGGCTTGTACCAAGGGCTCAGC |
| | ACAGCAACGAAGGACACATACGATGCGTTGCACATGCAAGCACT |
| | CCCGCCCCGC |
| CD-19 specific CAR, amino | MLLLVTSLLLCELPHPAFLLIPDIQMTQTTSSLSASLGDRVTISCRASQ |
| acid (SEQ ID NO: 2) | DISKYLNWYQQKPDGTVKLLIYHTSRLHSGVPSRFSGSGSGTDYSLT |
| | ISNLEQEDIATYFCQQGNTLPYTFGGGTKLEITGSTSGSGKPGSGEGS |
| | TKGEVKLQESGPGLVAPSQSLSVTCTVSGVSLPDYGVSWIRQPPRK |
| | GLEWLGVIWGSETTYYNSALKSRLTIIKDNSKSQVFLKMNSLQTDD |
| | TAIYYCAKHYYYGGSYAMDYWGQGTSVTVSSAAATTTPAPRPPTPA |
| | PTIASQPLSLRPEACRPAAGGAVHTRGLDFACDIYIWAPLAGTCGVL |
| | LLSLVITLYCKRGRKKLLYIFKQPFMRPVQTTQEEDGCSCRFPEEEEG |
| | GCELRVKFSRSADAPAYQQGQNQLYNELNLGRREEYDVLDKRRGR |
| | DPEMGGKPRRKNPQEGLYNELQKDKMAEAYSEIGMKGERRRGKG |
| | HDGLYQGLSTATKDTYDALHMQALPPR |
| Granzyme B shuttle 21-247, | AATGATTATAGGCGGACATGAAGCAAAGCCGCACTCCCGGCCGTA |
| nucleic acid (SEQ ID NO: | CATGGCCTACTTGATGATTTGGGATCAAAAATCCCTTAAAAGGTG |
| 3) | CGGAGGCTTCCTGATTCGCGATGATTTTGTACTGACTGCCGCCCA |
| | CTGCTGGGGCAGTTCTATAAACGTCACTCTTGGTGCACATAACAT |
| | CAAAGAGCAGGAACCTACACAACAATTCATACCCGTCAAGCGAC |
| | CAATTCCCCATCCTGCATATAATCCGAAAAACTTTTCTAATGACAT |
| | AATGCTGCTCCAACTTGAGAGGAAGGCAAAGCGGACGCGCGCC |
| | GTTCAACCCTTGCGACTCCCGTCAAACAAAGCCCAGGTCAAACC |
| | AGGACAAACGTGCTCAGTTGCGGGTTGGGGTCAAACAGCGCCCC |
| | TCGGCAAGCACTCACACACTCTTCAAGAAGTAAAAATGACGGTT |
| | CAAGAAGATCGCAAATGCGAGTCTGACCTGAGGCATTATTACGAT |
| | TCCACCATAGAGCTTTGTGTGGGTGATCCAGAGATTAAAAAGACT |
| | TCATTCAAAGGAGACAGTGGTGGACCTCTTGTGTGCAATAAAGT |
| | CGCTCAAGGGATCGTTTCATACGGTAGAAATAACGGCATGCCTCC |
| | TCGCGCTTGTACTAAAGTGTCATCCTTCGTTCATTGGATCAAAAA |
| | AACAATGAAAAGATAC |
| Granzyme B shuttle 21-247, | MIIGGHEAKPHSRPYMAYLMIWDQKSLKRCGGFLIRDDFVLTAAHC |
| amino acid (SEQ ID NO: 4) | WGSSINVTLGAHNIKEQEPTQQFIPVKRPIPHPAYNPKNFSNDIMLLQ |
| | LERKAKRTRAVQPLRLPSNKAQVKPGQTCSVAGWGQTAPLGKHSH |
| | TLQEVKMTVQEDRKCESDLRHYYDSTIELCVGDPEIKKTSFKGDSG |
| | GPLVCNKVAQGIVSYGRNNGMPPRACTKVSSFVHWIKKTMKRY |
| scFv binder for BCL2, | GAAGTACAACTCGTCGAGAGCGGCGGCGGCCCCGTGCAGCCAG |
| nucleic acid (SEQ ID NO: | GAGGTTCATTGCGGCTGTCATGTACTGGGAGTGGATTCTTCGGTA |
| 5) | TGTGGGTGAGGCAGCCAGGGAAGGGGCTTGAATGGGTATCAATC |
| | GGCACCTATGCGGACAGTGTGAAAGGCCGATTCAGCAGGGACAA |
| | CGCCAAGAATACCTTGTATCTTCAAATGAATCTTGAAGATACTGC |
| | GGTTTATTGCGCTAAGCCGGTCACAATACTTTTCGGGACGTGGAA |
| | GCGGTACGACTACTGGGGACAAGGGACGCAGGTCACAGTTAGTT |
| | CT |
| scFv binder for BCL2, amino | EVQLVESGGGPVQPGGSLRLSCTGSGFFGMWVRQPGKGLEWVSIG |
| acid (SEQ ID NO: 6) | TYADSVKGRFSRDNAKNTLYLQMNLEDTAVYCAKPVTILFGTWKR |
| | YDYWGQGTQVTVSS |
| Cereblon E3UL, nucleic acid | ATGGCCGGAGAGGGAGATCAACAGGATGCGGCGCATAACATGGG |
| (SEQ ID NO: 7) | AAACCACCTGCCTTTGCTTCCAGCCGAGTCAGAGGAGGAAGACG |
| | AGATGGAAGTAGAGGACCAGGATTCAAAAGAGGCTAAGAAACC |
| | AAACATCATCAACTTCGACACCAGTCTGCCTACTAGCCACACATA |
| | CCTGGGGGCTGACATGGAAGAGTTTCACGGGCGGACACTTCACG |
| | ACGACGATAGCTGTCAAGTTATCCCTGTCCTGCCCCAGGTCATGA |
| | TGATCCTTATTCCCGGGCAAACCCTCCCCCTGCAACTCTTCCACC |
| | CACAAGAGGTTAGCATGGTAAGAAACCTCATACAGAAGGATAGA |
| | ACATTCGCGGTGTTGGCTTATTCAAATGTACAGGAGAGGGAAGC |
| | ACAATTCGGTACGACTGCCGAAATATACGCATATAGAGAGGAGCA |
| | GGATTTTGGCATCGAAATAGTAAAGGTTAAGGCCATCGGTCGCCA |
| | ACGATTTAAGGTACTCGAACTGAGGACCCAGAGTGACGGGATTC |
| | AACAGGCCAAAGTCCAGATACTGCCCGAGTGTGTGTTGCCTTCA |
| | ACTATGTCAGCCGTTCAACTTGAATCTCTTAACAAGTGCCAAATC |
| | TTTCCGTCCAAACCAGTAAGCCGCGAAGACCAATGTTCTTATAAG |
| | TGGTGGCAAAAATATCAGAAGCGAAAGTTTCATTGCGCTAATCTT |
| | ACGTCATGGCCGCGGTGGCTTTATAGTCTTTACGACGCGGAAACT |
| | CTGATGGACAGGATTAAGAAACAGCTGCGAGAGTGGGACGAGA |
| | ATCTTAAAGATGATTCTCTCCCTTCAAATCCTATTGACTTTAGTTAC |
| | AGAGTCGCCGCTTGTTTGCCCATCGACGATGTGCTGAGAATCCAG |
| | CTGTTGAAAATCGGTTCAGCCATCCAGAGGTTGCGATGTGAACTT |
| | GATATAATGAATAAATGCACTAGTCTCTGCTGTAAGCAATGCCAGG |
| | AGACAGAAATTACAACGAAAAATGAAATTTTTTCCCTTAGCCTGT |
| | GTGGGCCTATGGCCGCTTATGTGAACCCGCACGGTTACGTGCACG |
| | AGACTCTGACCGTGTACAAGGCCTGCAACCTTAACTTGATTGGTC |
| | GACCATCAACAGAGCACAGCTGGTTTCCGGGGTACGCTTGGACC |
| | GTGGCACAATGTAAGATCTGCGCGAGTCATATTGGGTGGAAGTTC |
| | ACAGCGACTAAGAAGGATATGAGTCCGCAGAAGTTCTGGGGCTT |
| | GACACGCTCTGCTCTTCTGCCAACCATCCCAGATACGGAAGATGA |
| | AATTAGCCCAGACAAGGTAATTCTCTGCCTC |
| Cereblon E3UL, amino acid | MAGEGDQQDAAHNMGNHLPLLPAESEEEDEMEVEDQDSKEAKKP |
| (SEQ ID NO: 8) | NIINFDTSLPTSHTYLGADMEEFHGRTLHDDDSCQVIPVLPQVMMIL |
| | IPGQTLPLQLFHPQEVSMVRNLIQKDRTFAVLAYSNVQEREAQFGTT |
| | AEIYAYREEQDFGIEIVKVKAIGRQRFKVLELRTQSDGIQQAKVQILP |
| | ECVLPSTMSAVQLESLNKCQIFPSKPVSREDQCSYKWWQKYQKRK |
| | FHCANLTSWPRWLYSLYDAETLMDRIKKQLREWDENLKDDSLPSN |
| | PIDFSYRVAACLPIDDVLRIQLLKIGSAIQRLRCELDIMNKCTSLCCK |
| | QCQETEITTKNEIFSLSLCGPMAAYVNPHGYVHETLTVYKACNLNLI |
| | GRPSTEHSWFPGYAWTVAQCKICASHIGWKFTATKKDMSPQKFWG |
| | LTRSALLPTIPDTEDEISPDKVILCL |
| Full-length FP1, nucleic acid | ATGATTATTGGTGGACACGAAGCCAAGCCACACAGCCGACCATA |
| (SEQ ID NO: 9) | CATGGCATATCTTATGATCTGGGACCAGAAGAGCCTCAAGCGGTG |
| | CGGCGGATTCTTGATCAGAGACGACTTTGTGCTTACAGCTGCTCA |
| | TTGCTGGGGCTCCAGCATCAATGTCACACTCGGCGCCCACAATAT |
| | CAAAGAACAAGAACCAACGCAACAGTTTATCCCGGTCAAACGAC |
| | CCATACCCCATCCTGCCTACAACCCGAAGAACTTCAGCAATGACA |
| | TAATGCTGCTCCAACTCGAGCGGAAGGCGAAAAGGACTCGGGCC |
| | GTCCAACCACTCAGATTGCCCTCCAATAAAGCTCAGGTAAAACC |
| | AGGCCAAACGTGCTCTGTCGCAGGCTGGGGGCAAACAGCACCC |
| | CTTGGTAAGCATAGTCATACTTTGCAAGAAGTTAAAATGACAGTA |
| | CAGGAAGATCGAAAATGTGAATCTGACCTTCGCCATTATTACGAT |
| | TCCACCATTGAGCTGTGTGTAGGAGATCCAGAAATCAAGAAGAC |
| | TAGTTTTAAGGGCGATTCAGGAGGACCGCTGGTGTGCAATAAAGT |
| | GGCCCAGGGAATCGTTTCTTACGGAAGGAACAACGGAATGCCTC |
| | CGCGGGCATGCACTAAGGTGTCTTCCTTTGTACATTGGATTAAAA |
| | AGACAATGAAACGCTACGGTGGCGGCGGATCCGGGGGCGGCGGT |
| | TCTGGAGGGGGCGGCTCAGAAGTACAGCTTGTAGAATCAGGTGG |
| | CGGGCCGGTACAGCCCGGGGGGAGTCTTCGACTGTCTTGCACGG |
| | GGTCTGGCTTCTTCGGCATGTGGGTCAGGCAGCCAGGGAAAGGG |
| | CTTGAATGGGTGAGCATAGGAACTTATGCGGACAGTGTCAAAGG |
| | AAGATTCAGCCGAGATAACGCGAAGAACACTTTGTACCTCCAAA |
| | TGAATCTGGAAGACACCGCCGTGTACTGCGCCAAGCCGGTCACT |
| | ATCCTGTTCGGGACCTGGAAGAGGTATGATTACTGGGGACAAGG |
| | GACACAAGTAACCGTCAGCAGCGGCGGCGGGGGAAGCGGTGGT |
| | GGTGGGTCCGGAGGTGGCGGATCCATGGCTGGAGAGGGAGATCA |
| | ACAAGACGCAGCTCATAATATGGGAAATCATCTCCCCCTGCTGCC |
| | GGCAGAAAGCGAGGAAGAGGACGAGATGGAGGTGGAGGATCAA |
| | GATTCCAAAGAGGCTAAGAAGCCAAATATCATCAACTTCGATACT |
| | TCCCTGCCTACCTCACATACTTATCTTGGTGCTGATATGGAGGAGT |
| | TCCATGGTCGCACCTTGCATGATGATGATTCTTGCCAGGTAATTCC |
| | TGTGTTGCCGCAGGTGATGATGATACTTATCCCTGGTCAAACGTT |
| | GCCGTTGCAACTTTTCCACCCACAGGAAGTTTCTATGGTCAGGAA |
| | TCTCATCCAGAAGGACAGAACGTTTGCGGTTTTGGCGTATAGTAA |
| | CGTACAAGAGAGGGAAGCACAATTCGGTACGACTGCCGAGATAT |
| | ATGCTTATCGCGAGGAACAGGACTTCGGAATTGAAATAGTAAAGG |
| | TAAAAGCAATTGGGCGCCAACGGTTTAAAGTATTGGAGCTCAGG |
| | ACGCAAAGTGATGGCATCCAGCAGGCAAAGGTGCAGATATTGCC |
| | CGAATGTGTTCTTCCCTCTACGATGTCTGCCGTGCAGTTGGAATC |
| | CCTGAACAAGTGTCAAATTTTCCCCTCAAAACCAGTCAGCAGGG |
| | AGGATCAATGTAGTTATAAGTGGTGGCAGAAATATCAGAAGCGAA |
| | AGTTCCACTGCGCCAACCTGACAAGTTGGCCACGGTGGCTGTATT |
| | CTCTGTATGATGCGGAGACACTGATGGACAGAATCAAAAAACAA |
| | CTTAGGGAATGGGATGAAAACCTGAAAGACGACTCCCTTCCGTC |
| | CAACCCAATAGACTTTAGTTATCGCGTCGCTGCGTGCCTCCCTATC |
| | GATGACGTTCTTAGAATCCAACTCCTCAAGATCGGCAGCGCTATT |
| | CAGAGGCTGCGGTGTGAGCTGGATATAATGAATAAATGCACGAGT |
| | CTTTGTTGTAAACAGTGCCAAGAAACGGAGATAACCACAAAGAA |
| | TGAAATATTCAGTCTGTCTCTTTGCGGGCCGATGGCAGCATATGTG |
| | AATCCTCATGGGTACGTGCACGAGACGCTGACAGTCTACAAGGC |
| | GTGCAATCTCAACCTGATCGGAAGGCCATCCACGGAACACAGTT |
| | GGTTTCCCGGCTATGCATGGACAGTAGCGCAGTGTAAGATTTGTG |
| | CAAGCCATATAGGTTGGAAGTTCACCGCGACGAAGAAGGACATG |
| | TCTCCTCAGAAATTCTGGGGCCTCACACGCTCTGCACTTCTCCCG |
| | ACAATACCGGATACGGAGGACGAGATCTCCCCTGACAAAGTGAT |
| | CCTCTGCCTCGATTACGACGATGATGATAAG |
| Full-length FP1, amino acid | MIIGGHEAKPHSRPYMAYLMIWDQKSLKRCGGFLIRDDFVLTAAHC |
| (SEQ ID NO: 10) | WGSSINVTLGAHNIKEQEPTQQFIPVKRPIPHPAYNPKNFSNDIMLLQ |
| | LERKAKRTRAVQPLRLPSNKAQVKPGQTCSVAGWGQTAPLGKHSH |
| | TLQEVKMTVQEDRKCESDLRHYYDSTIELCVGDPEIKKTSFKGDSG |
| | GPLVCNKVAQGIVSYGRNNGMPPRACTKVSSFVHWIKKTMKRYGG |
| | GGSGGGGSGGGGSEVQLVESGGGPVQPGGSLRLSCTGSGFFGMWV |
| | RQPGKGLEWVSIGTYADSVKGRFSRDNAKNTLYLQMNLEDTAVYC |
| | AKPVTILFGTWKRYDYWGQGTQVTVSSGGGGSGGGGSGGGGSMA |
| | GEGDQQDAAHNMGNHLPLLPAESEEEDEMEVEDQDSKEAKKPNII |
| | NFDTSLPTSHTYLGADMEEFHGRTLHDDDSCQVIPVLPQVMMILIPG |
| | QTLPLQLFHPQEVSMVRNLIQKDRTFAVLAYSNVQEREAQFGTTAEI |
| | YAYREEQDFGIEIVKVKAIGRQRFKVLELRTQSDGIQQAKVQILPEC |
| | VLPSTMSAVQLESLNKCQIFPSKPVSREDQCSYKWWQKYQKRKFH |
| | CANLTSWPRWLYSLYDAETLMDRIKKQLREWDENLKDDSLPSNPID |
| | FSYRVAACLPIDDVLRIQLLKIGSAIQRLRCELDIMNKCTSLCCKQCQ |
| | ETEITTKNEIFSLSLCGPMAAYVNPHGYVHETLTVYKACNLNLIGRP |
| | STEHSWFPGYAWTVAQCKICASHIGWKFTATKKDMSPQKFWGLTRS |
| | ALLPTIPDTEDEISPDKVILCLDYDDDDK |

The CAR-T fusion coding sequence will be cloned into the XhoI site (2810) of the pLVX-EF1a-IRES-ZsGreen1 bicistronic lentiviral vector (Takara Bio USA, San Jose, CA; Cat. No. 632187). Following sequence confirmation of successful cloning by Sanger sequencing, the construct will be digested with KpnI to remove the nucleic acid sequence from nucleotide number 3289 to nucleotide number 4854. The bioProtac coding sequences with the Kpn1 truncated portion of the IRES sequence (nucleotide sequences between 3289 and 4216 of the pLVX-EF1a-IRES-ZsGreen1 plasmid at the 5′ end of the sequence and the WPRE sequence removed from the Kpn1 digestion at the 3′ end of the construct (4126 to 4854) will be ligated in and their sequences will be confirmed via Sanger sequencing.

### Lentiviral Vector Production

HEK 293T cells will be cultured in DMEM with 10% FBS until they are at 70-80% confluency. These cells will be transfected with the lentiviral vector discussed above, psPAX2, and pMD2.G (4:3:1 ratio) using Lipofectamine 3000. The cells will be incubated for 48-72 hours. The supernatant will be harvested, filtered through a 0.45 μm pore size filter, concentrated by ultracentrifugation, and the titer will be determined via p24 ELISA or flow cytometry.

### Isolation, Activation, and Transduction of Human T Cells

Peripheral blood mononuclear cells (PBMCs) will be isolated from donor blood via Ficoll-Paque centrifugation, and CD3+ T cells will be enriched using magnetic bead negative selection. The CD3+ T cells will be activated in X-Vivo 15 medium with 5-10% human serum, 50-100 IU/mL IL-2, and anti-CD3/CD28 Dynabeads (1:1 ratio) for 24-48 hours. The CD3+ T cells will be transduced on retronectin-coated plates with lentivirus and polybrene (6-8 μg/mL) via spinoculation (800×g, 60-90 min, 32° C.), then cultured 3-5 days post-transduction.

### Verification of Fusion Protein Expression and Functional Characterization

Protein expression will be analyzed by SDS-PAGE and Western blot using anti-granzyme B, anti-tag, and anti-E3 ligase antibodies. CAR expression and fusion protein localization will be confirmed via flow cytometry and confocal microscopy. The CD3+ T cells will be co-cultured with CD19+ Raji cells. Synapse formation and fusion protein release will be monitored via live-cell imaging and ELISA.

### Verification of Intracellular Uptake and Oncoprotein Degradation

The CD19+ cancer cells will be sorted post-co-culture using fluorescence-activated cells orting (FACS). Oncoprotein levels will be assessed by Western blot and qPCR. Losses will be visualized via immunofluorescence. Apoptosis will be quantified with Annexin V/PI staining and caspase-3/7 activity assays.

### In Vitro Cytotoxicity Testing

CAR-T cells will be co-cultured with luciferase-labeled CD19+ Raji cells at E:T ratios (1:1, 5:1, 10:1) for 16-24 hours. Viability will be measured via luminescence or MTS assay. A serial killing assay will be performed with periodic cancer cell addition. Sustained cytotoxicity will be assessed via luminescence and flow cytometry.

### In Vivo Xenograft Efficacy Studies

NSG mice will be injected with luciferase-labeled CD19+ Raji cells subcutaneously. Tumors will be allowed to reach 50-100 mm³. 1-5×10⁶ CAR-T cells will be administered intravenously, and tumor burden will be monitored via bioluminescence and calipers. BCL2 levels in harvested tumors will be assessed via immunohistochemistry and Western blot. The safety will be evaluated via cytokine levels and histopathology.

### Example 2: Gene Editing in Lymphocytes for Genetic Disorders

This example will outline a gene editing strategy where lymphocytes express a fusion protein with a Granzyme K shuttle, a TALE DNA-binding domain, and a FokI nuclease to correct pathogenic DNA sequences in genetic disorders, using homologous recombination with an AAV6-delivered donor template.

### Fusion Protein Design and Assembly

A fusion protein (FP2) will be designed with a Granzyme K shuttle (PCR-amplified from lymphocyte cDNA), a TALE domain targeting a specific pathogenic sequence (via RVD technology), and a FokI nuclease, linked by GGGGS repeats The fusion protein was assembled using Golden Gate cloning into a lentiviral vector, such as the one described in Example 1 (SEQ ID NO: 11); verify by Sanger sequencing and Western blot. The sequences for the components of this fusion protein are described in Table 2 below.

TABLE 2
Sequences of FP2 Components

| FP2 Component | Sequence |
|---|---|
| Granzyme K shuttle, nucleic | ATGACTAAGTTTTCTTCCTTTTCTCTGTTTTTCCTAATAGTTGGGG |
| acid (SEQ ID NO: 11) | CTTATATGACTCATGTGTGTTTCAATATGGAAATTATTGGAGGGAA |
| | AGAAGTGTCACCTCATTCCAGGCCATTTATGGCCTCCATCCAGTAT |
| | GGCGGACATCACGTTTGTGGAGGTGTTCTGATTGATCCACAGTGG |
| | GTGCTGACAGCAGCCCACTGCCAATATCGGTTTACCAAAGGCCA |
| | GTCTCCCACTGTGGTTTTAGGCGCACACTCTCTCTCAAAGAATGA |
| | GGCCTCCAAACAAACACTGGAGATCAAAAAATTTATACCATTCTC |
| | AAGAGTTACATCAGATCCTCAATCAAATGATATCATGCTGGTTAAG |
| | CTTCAAACAGCCGCAAAACTCAATAAACATGTCAAGATGCTCCA |
| | CATAAGATCCAAAACCTCTCTTAGATCTGGAACCAAATGCAAGGT |
| | TACTGGCTGGGGAGCCACCGATCCAGATTCATTAAGACCTTCTGA |
| | CACCCTGCGAGAAGTCACTGTTACTGTCCTAAGTCGAAAACTTTG |
| | CAACAGCCAAAGTTACTACAACGGCGACCCTTTTATCACCAAAG |
| | ACATGGTCTGTGCAGGAGATGCCAAAGGCCAGAAGGATTCCTGT |
| | AAGGGTGACTCAGGGGGCCCCTTGATCTGTAAAGGTGTCTTCCA |
| | CGCTATAGTCTCTGGAGGTCATGAATGTGGTGTTGCCACAAAGCC |
| | TGGAATCTACACCCTGTTAACCAAGAAATACCAGACTTGGATCAA |
| | AAGCAACCTTGTCCCGCCTCATACAAATTA |
| Granzyme K shuttle, amino | MTKFSSFSLFFLIVGAYMTHVCFNMEIIGGKEVSPHSRPFMASIQYG |
| acid (SEQ ID NO: 12) | GHHVCGGVLIDPQWVLTAAHCQYRFTKGQSPTVVLGAHSLSKNEA |
| | SKQTLEIKKFIPFSRVTSDPQSNDIMLVKLQTAAKLNKHVKMLHIRS |
| | KTSLRSGTKCKVTGWGATDPDSLRPSDTLREVTVTVLSRKLCNSQS |
| | YYNGDPFITKDMVCAGDAKGQKDSCKGDSGGPLICKGVFHAIVSG |
| | GHECGVATKPGIYTLLTKKYQTWIKSNLVPPHTN |
| TALE domain, nucleic acid | ATGGCCCCCAAGAAGAAGAGGAAGGTGGGCATCCACGGGGTAC |
| (SEQ ID NO: 13) | CTATGGTGGACTTGAGGACACTCGGTTATTCGCAACAGCAACAG |
| | GAGAAAATCAAGCCTAAGGTCAGGAGCACCGTCGCGCAACACC |
| | ACGAGGCGCTTGTGGGGCATGGCTTCACTCATGCGCATATTGTCG |
| | CGCTTTCACAGCACCCTGCGGCGCTTGGGACGGTGGCTGTCAAA |
| | TACCAAGATATGATTGCGGCCCTGCCCGAAGCCACGCACGAGGC |
| | AATTGTAGGGGTCGGTAAACAGTGGTCGGGAGCGCGAGCACTTG |
| | AGGCGCTGCTGACTGTGGCGGGTGAGCTTAGGGGGCCTCCGCTC |
| | CAGCTCGACACCGGGCAGCTGCTGAAGATCGCGAAGAGAGGGG |
| | GAGTAACAGCGGTAGAGGCAGTGCACGCCTGGCGCAATGCGCTC |
| | ACCGGGGCCCCCTTGAACCTGACCCCAGACCAGGTAGTCGCAAT |
| | CGCGAACAATAATGGGGGAAAGCAAGCCCTGGAAACCGTGCAA |
| | AGGTTGTTGCCGGTCCTTTGTCAAGACCACGGCCTTACACCGGA |
| | GCAAGTCGTGGCCATTGCAAATAATAACGGTGGCAAACAGGCTC |
| | TTGAGACGGTTCAGAGACTTCTCCCAGTTCTCTGTCAAGCCCACG |
| | GGCTGACTCCCGATCAAGTTGTAGCGATTGCGTCCAACGGTGGA |
| | GGGAAACAAGCATTGGAGACTGTCCAACGGCTCCTTCCCGTGTT |
| | GTGTCAAGCCCACGGTTTGACGCCTGCACAAGTGGTCGCCATCG |
| | CCAGCCATGATGGCGGTAAGCAGGCGCTGGAAACAGTACAGCGC |
| | CTGCTGCCTGTACTGTGCCAGGATCATGGACTGACCCCAGACCAG |
| | GTAGTCGCAATCGCGTCACATGACGGGGGAAAGCAAGCCCTGGA |
| | AACCGTGCAAAGGTTGTTGCCGGTCCTTTGTCAAGACCACGGCC |
| | TTACACCGG |
| TALE domain, amino acid | MAPKKKRKVGIHGVPMVDLRTLGYSQQQQEKIKPKVRSTVAQHHE |
| (SEQ ID NO: 14) | ALVGHGFTHAHIVALSQHPAALGTVAVKYQDMIAALPEATHEAIVG |
| | VGKQWSGARALEALLTVAGELRGPPLQLDTGQLLKIAKRGGVTAV |
| | EAVHAWRNALTGAPLNLTPDQVVAIANNNGGKQALETVQRLLPVL |
| | CQDHGLTPEQVVAIANNNGGKQALETVQRLLPVLCQAHGLTPDQV |
| | VAIASNGGGKQALETVQRLLPVLCQAHGLTPAQVVAIASHDGGKQA |
| | LETVQRLLPVLCQDHGLTPDQVVAIASHDGGKQALETVQRLLPVLC |
| | QDHGLTP |
| Full-length FP2, nucleic acid | ATGACTAAGTTTTCTTCCTTTTCTCTGTTTTTCCTAATAGTTGGGG |
| (SEQ ID NO: 15) | CTTATATGACTCATGTGTGTTTCAATATGGAAATTATTGGAGGGAA |
| | AGAAGTGTCACCTCATTCCAGGCCATTTATGGCCTCCATCCAGTAT |
| | GGCGGACATCACGTTTGTGGAGGTGTTCTGATTGATCCACAGTGG |
| | GTGCTGACAGCAGCCCACTGCCAATATCGGTTTACCAAAGGCCA |
| | GTCTCCCACTGTGGTTTTAGGCGCACACTCTCTCTCAAAGAATGA |
| | GGCCTCCAAACAAACACTGGAGATCAAAAAATTTATACCATTCTC |
| | AAGAGTTACATCAGATCCTCAATCAAATGATATCATGCTGGTTAAG |
| | CTTCAAACAGCCGCAAAACTCAATAAACATGTCAAGATGCTCCA |
| | CATAAGATCCAAAACCTCTCTTAGATCTGGAACCAAATGCAAGGT |
| | TACTGGCTGGGGAGCCACCGATCCAGATTCATTAAGACCTTCTGA |
| | CACCCTGCGAGAAGTCACTGTTACTGTCCTAAGTCGAAAACTTTG |
| | CAACAGCCAAAGTTACTACAACGGCGACCCTTTTATCACCAAAG |
| | ACATGGTCTGTGCAGGAGATGCCAAAGGCCAGAAGGATTCCTGT |
| | AAGGGTGACTCAGGGGGCCCCTTGATCTGTAAAGGTGTCTTCCA |
| | CGCTATAGTCTCTGGAGGTCATGAATGTGGTGTTGCCACAAAGCC |
| | TGGAATCTACACCCTGTTAACCAAGAAATACCAGACTTGGATCAA |
| | AAGCAACCTTGTCCCGCCTCATACAAATGGTGGTGGTGGTAGTAT |
| | GGCCCCCAAGAAGAAGAGGAAGGTGGGCATCCACGGGGTACCT |
| | ATGGTGGACTTGAGGACACTCGGTTATTCGCAACAGCAACAGGA |
| | GAAAATCAAGCCTAAGGTCAGGAGCACCGTCGCGCAACACCACG |
| | AGGCGCTTGTGGGGCATGGCTTCACTCATGCGCATATTGTCGCGC |
| | TTTCACAGCACCCTGCGGCGCTTGGGACGGTGGCTGTCAAATAC |
| | CAAGATATGATTGCGGCCCTGCCCGAAGCCACGCACGAGGCAAT |
| | TGTAGGGGTCGGTAAACAGTGGTCGGGAGCGCGAGCACTTGAGG |
| | CGCTGCTGACTGTGGCGGGTGAGCTTAGGGGGCCTCCGCTCCAG |
| | CTCGACACCGGGCAGCTGCTGAAGATCGCGAAGAGAGGGGGAG |
| | TAACAGCGGTAGAGGCAGTGCACGCCTGGCGCAATGCGCTCACC |
| | GGGGCCCCCTTGAACCTGACCCCAGACCAGGTAGTCGCAATCGC |
| | GAACAATAATGGGGGAAAGCAAGCCCTGGAAACCGTGCAAAGG |
| | TTGTTGCCGGTCCTTTGTCAAGACCACGGCCTTACACCGGAGCA |
| | AGTCGTGGCCATTGCAAATAATAACGGTGGCAAACAGGCTCTTG |
| | AGACGGTTCAGAGACTTCTCCCAGTTCTCTGTCAAGCCCACGGG |
| | CTGACTCCCGATCAAGTTGTAGCGATTGCGTCCAACGGTGGAGG |
| | GAAACAAGCATTGGAGACTGTCCAACGGCTCCTTCCCGTGTTGT |
| | GTCAAGCCCACGGTTTGACGCCTGCACAAGTGGTCGCCATCGCC |
| | AGCCATGATGGCGGTAAGCAGGCGCTGGAAACAGTACAGCGCCT |
| | GCTGCCTGTACTGTGCCAGGATCATGGACTGACCCCAGACCAGG |
| | TAGTCGCAATCGCGTCACATGACGGGGGAAAGCAAGCCCTGGAA |
| | ACCGTGCAAAGGTTGTTGCCGGTCCTTTGTCAAGACCACGGCCT |
| | TACACCGG |
| Full-length FP2, amino acid | MTKFSSFSLFFLIVGAYMTHVCFNMEIIGGKEVSPHSRPFMASIQYG |
| (SEQ ID NO: 16) | GHHVCGGVLIDPQWVLTAAHCQYRFTKGQSPTVVLGAHSLSKNEA |
| | SKQTLEIKKFIPFSRVTSDPQSNDIMLVKLQTAAKLNKHVKMLHIRS |
| | KTSLRSGTKCKVTGWGATDPDSLRPSDTLREVTVTVLSRKLCNSQS |
| | YYNGDPFITKDMVCAGDAKGQKDSCKGDSGGPLICKGVFHAIVSG |
| | GHECGVATKPGIYTLLTKKYQTWIKSNLVPPHTNGGGGSMAPKKKR |
| | KVGIHGVPMVDLRTLGYSQQQQEKIKPKVRSTVAQHHEALVGHGF |
| | THAHIVALSQHPAALGTVAVKYQDMIAALPEATHEAIVGVGKQWSG |
| | ARALEALLTVAGELRGPPLQLDTGQLLKIAKRGGVTAVEAVHAWRN |
| | ALTGAPLNLTPDQVVAIANNNGGKQALETVQRLLPVLCQDHGLTPE |
| | QVVAIANNNGGKQALETVQRLLPVLCQAHGLTPDQVVAIASNGGG |
| | KQALETVQRLLPVLCQAHGLTPAQVVAIASHDGGKQALETVQRLLP |
| | VLCQDHGLTPDQVVAIASHDGGKQALETVQRLLPVLCQDHGLTP |

The full-length fusion construct will be cloned into the XhoI site (2810) of the pLVX-EF1a-IRES-ZSGreen1 bicistronic lentiviral vector (Takara Bio USA, San Jose, CA; Cat. No. 632187). HEK 293T cells will be cultured in DMEM with 10% FBS until they are at 70-80% confluency. These cells will be transfected with the lentiviral vector discussed above, psPAX2, and pMD2.G (4:3:1 ratio) using Lipofectamine 3000 (ThermoFisher Scientific, Waltham, MA). The cells will be incubated for 48-72 hours. The supernatant will be harvested, filtered through a 0.45 μm pore size filter, concentrated by ultracentrifugation, and the titer will be determined via p24 ELISA or flow cytometry.

### Isolation, Activation, and Transduction of Lymphocytes

Following sequence confirmation, the construct will be transduced into T lymphocytes isolated from blood via leukapheresis, density gradient centrifugation, and immunomagnetic sorting. The T lymphocytes will be activated in RPMI 1640 with 10% FBS, IL-2, IL-7, IL-15, and anti-CD3/CD28 Dynabeads (ThermoFisher Scientific, Waltham, MA) for 40-48 hours. The T lymphocytes will be transduced with the lentiviral particles described above.

### In Vitro Validation of Gene Editing

Fusion protein expression and nuclear localization will be confirmed via flow cytometry and immunofluorescence with domain-specific antibodies. TALE binding will be assessed via ChIP-qPCR. DSBs will be measured via T7EI assay or deep sequencing. HDR efficiency will be quantified via junction-specific PCR and sequencing. A GFP reporter will be used if included.

### Ex Vivo Expansion and Functional Testing

Edited lymphocytes were expanded with anti-CD3/CD28 beads, IL-2, and IL-15. CD3/CD8 markers will be marked via flow cytometry. The edited lymphocytes will be co-cultured with target cells. Gene correction will be assessed via PCR/sequencing and functional restoration via viability/apoptosis assays.

### In Vivo Administration and Therapeutic Efficiency

The edited lymphocytes will be administered to NSG mice engrafted with target cells, and they will be tracked via bioluminescence and blood sampling. HDR efficiency and phenotype improvement in tissues will be measured via genomic analysis. Safety will be assessed for GvHD or off-target effects.

### Example 3: Inhibition of Viral Replication

This example will demonstrate inhibition of viral replication by engineering NK cells to express a fusion protein with a granulysin shuttle, an affibody binder for viral protein VP1, and a phosphatase effector to dephosphorylate the viral protein, reducing infectivity.

### Construction of Shuttle-Binder-Effector Fusion Protein

A fusion protein (FP3) will be designed with granulysin (amino acids 31-138), an scFV from HIV RT, and SPOP E3UL, linked by (GGGGS)₃, with a FLAG tag, as described in Table 3. This fusion protein will be cloned into the XhoI site (2810) of the pLVX-EF1a-IRES-ZsGreen1 biscistronic lentiviral vector (Takara Bio USA, San Jose, CA; Cat. No. 632187). HEK 293T cells will be cultured in DMEM with 1000 FBS until they are at 70-80%0 confluency. These cells will be transfected with the lentiviral vector discussed above, psPAX2, and pMD2.G (4:3:1 ratio) using Lipofectamine 3000 (ThermoFisher Scientific, Waltham, MA). The cells will be incubated for 48-72 hours. The supernatant will be harvested, filtered through a 0.45 μm pore size filter, concentrated by ultracentrifugation, and the titer will be determined via p24 ELISA or flow cytometry.

TABLE 3
Sequences of FP3 Components

| FP3 Component | Sequence |
|---|---|
| Granulysin amino acid | CTGGCTCGAGCTCATCTGCGGGATGAAGAGAAGTCATGCCCATG |
| residues 31-145, nucleic acid | CTTGGCTCAGGAAGGTCCGCAGGGAGACTTGCTCACTAAGACCC |
| (SEQ ID NO: 17) | AGGAGTTGGGTCGAGACTATCGCACGTGTCTCACAATAGTGCAG |
| | AAACTCAAGAAAATGGTCGACAAACCGACACAGCGCAGTGTCA |
| | GTAACGCTGCTACGCGAGTATGTCGAACCGGGCGGAGTCGGTGG |
| | CGGGATGTGTGCCGAAATTTCATGCGAAGGTATCAATCCCGAGTC |
| | ACGCAGGGCCTGGTGGCCGGTGAAACCGCCCAACAGATTTGCGA |
| | GGACTTGCGACTCTGCATACCCTCCACGGGTCCACTC |
| Granulysin amino acid | LARAHLRDEEKSCPCLAQEGPQGDLLTKTQELGRDYRTCLTIVQKL |
| residues 31-145, amino acid | KKMVDKPTQRSVSNAATRVCRTGRSRWRDVCRNFMRRYQSRVTQ |
| (SEQ ID NO: 18) | GLVAGETAQQICEDLRLCIPSTGPL |
| scFv binder region for HIV | ATGGCTCAAGTTCAACTCCAGCAATCAGGCCCAGGTCTTGTTAAA |
| RT, nucleic acid (SEQ ID | CCCTCCCAGACCCTCTCTCTCACCTGTGCTATTTCCGGCGACTCT |
| NO: 19) | GTCAGTAGCAACTCCGCCGCCTGGAATTGGATCAGGCAATCTCCT |
| | AGTCGGGGTTTGGAATGGTTGGGGAGAACTTACTACCGATCCAA |
| | GTGGTACAACGACTATGCAGTTTCTGTCAAGAGTCGAATCACAAT |
| | CAACCCAGATACTTCCAAGAACCAATTCTCTTTGCAGCTGAATAG |
| | TGTAACACCCGAAGATACCGCCGTTTATTATTGCGCCCGCGCCTG |
| | GGGCCCTCTCTTTGACTATTGGGGACAGGGCACATTGGTAACTGT |
| | GTCCTCTGGTGGAGGGGGGAGTGGAGGGGGTGGCTCAGGCGGT |
| | AGTGCGCTTTCCTCTGAGCTCACCCAGGACCCCGCCGTGAGCGT |
| | AGCTTTGGGACAGACAGTCCGAATCACATGTCAGGGCGATTCAC |
| | TGAGAAGCTATTACGCCAGCTGGTACCAGCAGAAACCTGGACAG |
| | GCCCCAGTTCTTGTTATCTACGGTAAAAATAATAGACCATCTGGTA |
| | TCCCCGACAGGTTCTCTGGCTCCTCTAGTTCAGGGAACACCGCAT |
| | CACTTACAATCACCGGCGCACAGGCTGAAGATGAGGCTGACTATT |
| | ACTGCAACTCCAGAGACTCCAGCGGAGCGATCGTGTTCGGTGGT |
| | GGGACGAAACTGACGGTTTTGGGG |
| scFv binder region for HIV | MAQVQLQQSGPGLVKPSQTLSLTCAISGDSVSSNSAAWNWIRQSPS |
| RT, amino acid (SEQ ID | RGLEWLGRTYYRSKWYNDYAVSVKSRITINPDTSKNQFSLQLNSVT |
| NO: 20) | PEDTAVYYCARAWGPLFDYWGQGTLVTVSSGGGGSGGGGSGGS |
| | ALSSELTQDPAVSVALGQTVRITCQGDSLRSYYASWYQQKPGQAPV |
| | LVIYGKNNRPSGIPDRFSGSSSSGNTASLTITGAQAEDEADYYCNSR |
| | DSSGAIVFGGGTKLTVLG |
| SPOP E3UL, nucleic acid | GTCAACATTAGCGGCCAGAACACCATGAACATGGTAAAGGTCCC |
| (SEQ ID NO: 21) | TGAATGTCGGTTGGCTGATGAATTGGGCGGACTGTGGGAGAATTC |
| | CAGGTTTACTGACTGTTGTCTTTGTGTGGCAGGGCAAGAATTCCA |
| | GGCGCACAAGGCCATTCTCGCTGCTCGGAGCCCAGTTTTCTCAG |
| | CCATGTTTGAGCACGAGATGGAGGAGTCTAAAAAAAATCGGGTA |
| | GAAATAAACGACGTTGAACCAGAAGTCTTTAAAGAAATGATGTG |
| | CTTCATATATACCGGCAAGGCACCAAATCTCGACAAAATGGCGGA |
| | CGATTTGCTTGCTGCGGCTGATAAATATGCCCTCGAACGGTTGAA |
| | GGTGATGTGCGAGGACGCCCTTTGTTCTAATCTTAGCGTCGAAAA |
| | CGCGGCTGAAATCCTTATTCTGGCGGACCTTCATTCCGCCGACCA |
| | ACTCAAGACCCAAGCCGTCGACTTCATAAACTATCATGCTTCAGA |
| | TGTGCTGGAAACCAGCGGCTGGAAGAGTATGGTGGTAAGTCACC |
| | CCCATCTTGTTGCGGAGGCATACCGAAGTCTTGCAAGCGCGCAAT |
| | GCCCATTTCTGGGACCACCACGCAAGCGACTCAAGCAAAGCGGG |
| | AGTGGGGCAACCAATTTTAGTCTGTTGAAACAGGCCGGAGATGT |
| | GGAAGAGAACCCAGGGCCTATGGTATCCAAAGGAGAGGAAGATA |
| | ACATGGCCATAATCAAAGAGTTCATGAGATTCAAGGTCCAC |
| SPOP E3UL, amino acid | VNISGQNTMNMVKVPECRLADELGGLWENSRFTDCCLCVAGQEFQ |
| (SEQ ID NO: 22) | AHKAILAARSPVFSAMFEHEMEESKKNRVEINDVEPEVFKEMMCFI |
| | YTGKAPNLDKMADDLLAAADKYALERLKVMCEDALCSNLSVENA |
| | AEILILADLHSADQLKTQAVDFINYHASDVLETSGWKSMVVSHPHL |
| | VAEAYRSLASAQCPFLGPPRKRLKQSGSGATNFSLLKQAGDVEENP |
| | GPMVSKGEEDNMAIIKEFMRFKVH |
| Full-length FP3, nucleic acid | CTTGCCAGGGCTCATCTCCGGGACGAGGAAAAAAGCTGTCCTTG |
| (SEQ ID NO: 23) | TTTGGCGCAGGAGGGACCCCAAGGCGACCTCTTGACAAAAACTC |
| | AGGAACTCGGCCGAGATTACCGGACATGTCTGACCATTGTGCAG |
| | AAGTTGAAAAAGATGGTCGATAAACCGACACAAAGGAGCGTGA |
| | GTAATGCCGCAACTAGAGTATGCAGAACGGGACGCAGCCGCTGG |
| | CGAGACGTTTGCCGCAACTTTATGAGACGGTACCAAAGTCGGGT |
| | AACCCAGGGTCTCGTCGCGGGCGAAACGGCCCAACAGATATGTG |
| | AAGATCTGCGACTCTGCATCCCCAGCACAGGTCCTCTTGGTGGCG |
| | GCGGAAGTGGGGGCGGTGGCAGTGGGGGAGGGGGTTCTATGGC |
| | TCAGGTGCAACTCCAGCAAAGCGGTCCAGGGTTGGTAAAGCCAT |
| | CTCAAACGCTGTCTCTTACTTGTGCTATTAGTGGGGATTCAGTTTC |
| | AAGCAATTCCGCAGCTTGGAACTGGATTAGACAATCACCTAGCCG |
| | GGGTTTGGAGTGGCTCGGGCGCACCTATTATCGGTCAAAGTGGTA |
| | TAACGATTACGCAGTCAGCGTTAAAAGCCGCATTACAATAAACCC |
| | GGATACATCCAAGAACCAGTTTTCTCTGCAGCTCAACTCTGTGAC |
| | GCCCGAGGACACTGCCGTCTATTATTGTGCTAGGGCGTGGGGACC |
| | GCTCTTCGATTACTGGGGCCAGGGAACATTGGTTACCGTATCAAG |
| | CGGGGGCGGGGGCTCAGGAGGAGGCGGCTCTGGAGGGTCAGCA |
| | CTTAGCTCTGAGCTGACGCAGGATCCTGCTGTATCAGTTGCACTT |
| | GGGCAGACTGTACGGATAACCTGCCAAGGGGATAGTCTGCGCTC |
| | TTACTACGCGTCATGGTATCAGCAAAAACCGGGTCAAGCACCTGT |
| | CCTGGTTATATACGGGAAGAATAATAGACCGTCAGGTATTCCTGAT |
| | CGATTCAGCGGTAGCTCCAGTAGCGGTAACACAGCTAGTCTGACT |
| | ATAACAGGTGCTCAAGCCGAGGATGAAGCTGATTATTATTGTAAC |
| | TCACGGGACAGCAGTGGGGCGATAGTCTTCGGCGGGGGCACGAA |
| | GTTGACGGTTCTCGGCGGCGGTGGCGGTTCTGGTGGAGGCGGGT |
| | CTGGAGGCGGAGGTTCAGTAAACATAAGCGGCCAAAATACTATG |
| | AACATGGTGAAAGTACCAGAATGCAGGTTGGCGGATGAGTTGGG |
| | GGGCCTGTGGGAGAATTCAAGATTCACCGACTGCTGTCTGTGCGT |
| | CGCTGGACAGGAATTCCAAGCGCATAAGGCGATACTGGCGGCAC |
| | GAAGTCCTGTCTTCAGCGCGATGTTTGAACATGAGATGGAAGAAT |
| | CTAAAAAAAATAGAGTCGAAATTAATGACGTTGAGCCAGAAGTG |
| | TTCAAAGAGATGATGTGCTTTATCTATACAGGAAAGGCACCTAAC |
| | CTTGATAAGATGGCAGATGACCTGCTTGCTGCTGCTGATAAGTAC |
| | GCGCTGGAGCGGTTGAAAGTCATGTGCGAGGATGCATTGTGTTC |
| | AAACCTTTCCGTCGAGAACGCCGCAGAAATCCTGATTCTCGCCG |
| | ATCTGCACAGCGCTGATCAGCTTAAGACTCAGGCAGTAGATTTTA |
| | TCAACTACCATGCCTCCGATGTGCTCGAGACATCAGGATGGAAGT |
| | CAATGGTAGTTTCTCATCCACATCTCGTAGCTGAGGCATATAGATC |
| | TCTGGCTTCAGCACAATGTCCATTCCTTGGACCCCCAAGAAAGCG |
| | ACTTAAGCAAAGCGGGTCAGGAGCCACCAATTTTAGCTTGTTGA |
| | AGCAAGCAGGAGACGTCGAGGAAAATCCTGGTCCGATGGTTAGC |
| | AAAGGTGAAGAGGACAATATGGCGATTATCAAAGAATTCATGAG |
| | ATTCAAAGTGCATGATTATAAAGACGATGATGATAAG |
| Full-length FP3, amino acid | LARAHLRDEEKSCPCLAQEGPQGDLLTKTQELGRDYRTCLTIVQKL |
| (SEQ ID NO: 24) | KKMVDKPTQRSVSNAATRVCRTGRSRWRDVCRNFMRRYQSRVTQ |
| | GLVAGETAQQICEDLRLCIPSTGPLGGGGSGGGGSGGGGSMAQVQL |
| | QQSGPGLVKPSQTLSLTCAISGDSVSSNSAAWNWIRQSPSRGLEWLG |
| | RTYYRSKWYNDYAVSVKSRITINPDTSKNQFSLQLNSVTPEDTAVYY |
| | CARAWGPLFDYWGQGTLVTVSSGGGGSGGGGSGGSALSSELTQDP |
| | AVSVALGQTVRITCQGDSLRSYYASWYQQKPGQAPVLVIYGKNNRP |
| | SGIPDRFSGSSSSGNTASLTITGAQAEDEADYYCNSRDSSGAIVFGGG |
| | TKLTVLGGGGGSGGGGSGGGGSVNISGQNTMNMVKVPECRLADE |
| | LGGLWENSRFTDCCLCVAGQEFQAHKAILAARSPVFSAMFEHEMEE |
| | SKKNRVEINDVEPEVFKEMMCFIYTGKAPNLDKMADDLLAAADKY |
| | ALERLKVMCEDALCSNLSVENAAEILILADLHSADQLKTQAVDFIN |
| | YHASDVLETSGWKSMVVSHPHLVAEAYRSLASAQCPFLGPPRKRLK |
| | QSGSGATNFSLLKQAGDVEENPGPMVSKGEEDNMAIIKEFMRFKV |
| | HDYKDDDDK |

### Genetic Modification of Natural Killer Cells

NK cells will be isolated from PBMCs via negative magnetic selection. These NK cells will be cultured in NK MACS medium with 5% serum, 500 U/mL IL-2, and 50 ng/mL IL-15. The NK cells will be transduced after 72 hours of activation with lentivirus (MOI 20) on RetroNectin plates (Takara Bio USA, San Jose, CA) with 6 μg/mL polybrene. The NK cells will be expanded for 7 days.

### Characteristics of Modified Natural Killer Cells

Fusion protein expression will be confirmed via flow cytometry (anti-FLAG) and Western blot. Granule localization will be verified via immunofluorescence. The cytotoxicity of the modified NK cells will be tested against K562 cells using a chromium-51 release assay.

### In Vitro Viral Inhibition Assays

A549 cells will be infected with RSV-GFP (MOI 0.1) for 4 hours. NK cells will be added in the following E:T ratios (1:1, 5:1, 10:1). Viral replication will be measured via GFP fluorescence, plaque assays, and qRT-PCR. VP1 phosphorylation will be assessed via Western blot.

### Mechanistic Studies

Okadaic acid will be used to inhibit phosphatase and confirm the mechanism of action for the NK cells. Immunoprecipitation will be performed to verify affibody-VP1 binding. Time-course experiments will be conducted to study delivery and dephosphorylation kinetics via immunofluorescence.

It is to be further understood that like numerals in the drawings represent like elements through the several figures, and that not all components and/or steps described and illustrated with reference to the figures are required for all embodiments or arrangements. Further, the terminology used herein is for the purpose of describing particular embodiments only and is not intended to be limiting of the described systems and methods. As used herein, the singular forms “a”, “an” and “the” are intended to include the plural forms as well, unless the context clearly indicates otherwise. It will be further understood that the terms ““including,” “comprising,” or “having,” “containing,” “involving,” and variations thereof herein, when used in this specification, specify the presence of stated features, integers, steps, operations, elements, and/or components, but do not preclude the presence or addition of one or more other features, integers, steps, operations, elements, components, and/or groups thereof.

It should be noted that use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Notably, the figures and examples above are not meant to limit the scope of the present disclosure to a single implementation, as other implementations are possible by way of interchange of some or all the described or illustrated elements. Moreover, where certain elements of the present disclosure can be partially or fully implemented using known components, only those portions of such known components that are necessary for an understanding of the present disclosure are described, and detailed descriptions of other portions of such known components are omitted so as not to obscure the disclosure. In the present specification, an implementation showing a singular component should not necessarily be limited to other implementations including a plurality of the same component, and vice-versa, unless explicitly stated otherwise herein. Moreover, applicants do not intend for any term in the specification or claims to be ascribed an uncommon or special meaning unless explicitly set forth as such. Further, the present disclosure encompasses present and future known equivalents to the known components referred to herein by way of illustration.

The foregoing description of the specific implementations will so fully reveal the general nature of the disclosure that others can, by applying knowledge within the skill of the relevant art(s), readily modify and/or adapt for various applications such specific implementations, without undue experimentation, without departing from the general concept of the present disclosure. Such adaptations and modifications are therefore intended to be within the meaning and range of equivalents of the disclosed implementations, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s). It is to be understood that dimensions discussed or shown are drawings are shown accordingly to one example and other dimensions can be used without departing from the disclosure.

The subject matter described above is provided by way of illustration only and should not be construed as limiting. Various modifications and changes can be made to the subject matter described herein without following the example embodiments and applications illustrated and described, and without departing from the true spirit and scope of the systems and methods encompassed by the present disclosure, which is defined by the set of recitations in the following claims and by structures and functions or steps which are equivalent to these recitations.

### REFERENCES

1. Baginska et al. PNAS. 2013 Oct. 22; 110(43):17450-5.
2. Bekes M et al. Nature Reviews Drug Discovery. 2022 March; 21(3):181-200.
3. Hlongwane et al. Biomedicines. 2018 Jun. 20; 6(2):72.
4. PCT International Patent Application Publication No. WO 2015/157864, published Oct. 10, 2015 (Holt et al.).
5. Hughes et al. Drug Discovery Today. 2021 Dec. 1; 26(12):2889-97
6. Lipoviek. Design & Selection. 2011 Jan. 1; 24(1-2):3-9.
7. Miersch S, Sidhu S S. F1000Research. 2016; 5.
8. Oberoi et al. PLoS One. 2013 Apr. 3; 8(4):e61267.
9. Woodsworth et al. Molecular Therapy—Methods & Clinical Development. 2017 Dec. 15; 7:132-45.
10. Lim et al. PNAS 2020 March 2; 117(11): 5791-5800.
11. U.S. Patent Application Publication No. 2023/0024904, published Jan. 26, 2023 (Mashiach et al.).
12. Restifo et al, Nature Reviews Immunology, 12: 269-281 (2011).
13. Lupton S. D. et al., Mol. and Cell Biol., 11:6 (1991).
14. Riddell et al., Human Gene Therapy 3:319-338 (1992).
15. PCT International Application Publication No. WO 1992/008796, published May 29, 1992 (Lupton).
16. PCT International Application Publication No. WO 1994/028143, published Dec. 8, 1994 (Lupton).
17. Dudley et al, J. Immunotherapy, 26(4): 332-342 (2003)
18. Dudley et al, Semin Oncol., 34(6): 524-531 (2007)
19. U.S. Pat. No. 6,040,177, issued Mar. 21, 2000 (Riddell et al.).
20. Kim et al, Nature Reviews Genetics, 15: 321-334 (2014).
21. Gaj et al, Trends Biotechnology, 31(7): 397-405 (2013).
22. Hsu et al, Cell, 157: 1262-1278 (2014).
23. Sander et al, Nature Biotechnology, 32(4): 347-355 (2014).
24. June et al, Nature Reviews Immunology, 9: 704-716 (2009).
25. Schmidt et al, Biotechnology J, 10: 258-272 (2015).
26. Mengstie, Front. Bioeng. Biotechnology, 12 May 2022:10:895713.
27. Halene et al, Blood, 94: 3349-3357 (1999).
28. Liechtenstein et al, Cancers, 5: 815-837 (2013).
29. U.S. Pat. No. 9,678,061, issued Jun. 13, 2017 (Dornmair et al.).
30. Breckpot et al., Gene Therapy, 14: 847-862 (2007).
31. U.S. Patent Application Publication No. 2001/0007659, published Jul. 12, 2001 (Wong-Staal et al.).
32. U.S. Pat. No. 8,951,535, issued Feb. 10, 2015 (Bauche and Sarry).
33. Lupton S. D., et al, Mol. and Cell. Biology, 11:3374-3378 (1991).
34. Coughlin et al, Blood, 103(6): 2046-2053 (2004).
35. Dull et al, J. Virol., 72(11): 8463-8471 (1998).
36. Naldini et al, Science, 272(5259): 263-267 (1996).
37. PCT International Application Publication No. WO 2015/157864, published Oct. 22, 2015 (Holt et al.).
38. Prager and Watzl, J. Leukoc. Biol. 105: 1319-1329 (2019).

What is claimed is:

1. A chimeric shuttle-binder-effector fusion protein, wherein:
the shuttle domain of said fusion protein comprises all a protein component of a lytic granule or a portion thereof;
the binder domain of said fusion protein comprises all or part of a protein epitope binder, a sequence-dependent DNA-binding protein, or a sequence-dependent RNA-binding protein;
the effector domain of said fusion protein comprises a protein selected to have a predetermined effect on a target molecule, wherein the effector domain comprises a nuclease, kinase, ubiquitin ligase, or a phosphatase; and
the domains are connected to one another by flexible linker sequences.

2. (canceled)

3. The fusion protein of claim 1, wherein the binder domain comprises a sequence-dependent DNA-binding domain, wherein said sequence-dependent DNA-binding protein is a transcription activator-like effector (TALE) protein, or a zinc-finger (ZF) protein).

4. The fusion protein of claim 1, wherein the binder domain comprises a protein epitope binder, wherein said protein epitope binder comprises a designed ankyrin repeat protein (DARPin), affibody, monobody, single-chain variable fragment (scFv), Fab, or a heavy chain antibody (HCab), further wherein the protein epitope binder may cause inhibition or activation of the target molecule.

5. (canceled)

6. (canceled)

7. The fusion protein of claim 1, wherein the shuttle domain of the fusion protein comprises a granzyme, the binder domain of the fusion protein comprises a protein epitope binder, and the effector domain of the fusion protein comprises a ubiquitin ligase or protease.

8. The fusion protein of claim 1, wherein the shuttle domain of the fusion protein consists of a granzyme, the binder domain of the fusion protein comprises a sequence-specific DNA-binding domain or RNA-binding domain, and the effector domain of the fusion protein comprises a nuclease.

9. A method of delivering a shuttle-binder-effector fusion protein into a target cell, the method comprising:
genetically modifying a lymphocyte or a stem cell that can be activated to become a lymphocyte to create a modified lymphocyte that expresses said fusion protein, said fusion protein comprising a shuttle domain, a binder domain, and an effector domain, wherein the domains are connected to each other by flexible linker sequences, wherein:
the shuttle domain of the shuttle-binder-effector fusion protein comprises a protein component of a lytic granule or a portion thereof,
the binder domain of said fusion protein comprises all or part of a protein epitope binder, a sequence-dependent DNA-binding protein, or a sequence-dependent RNA-binding protein, and
the effector domain of the shuttle-binder-effector fusion protein comprises a nuclease, kinase, ubiquitin ligase, or a phosphatase;
creating one or more delivery cells from the modified lymphocyte;
delivering the shuttle-binder-effector protein into the target cells via contact with the delivery cells.

10. (canceled)

11. (canceled)

12. The method of claim 9, wherein said lymphocyte is further genetically modified to attenuate or eliminate expression of endogenous cytotoxic effector mechanisms.

13. The method of claim 12, wherein the endogenous cytotoxic effector mechanisms being attenuated or eliminated comprise granzymes, Fas/FasL, and TRAIL pathways.

14. (canceled)

15. The method of claim 9, wherein the binder domain of the shuttle-binder effector fusion protein comprises a sequence-dependent DNA-binding protein, wherein the sequence-dependent DNA-binding protein is a transcription activator-like effector (TALE) protein, or a zinc-finger (ZF) protein.

16. The method of claim 9, wherein the binder domain of the shuttle-binder effector fusion protein comprises a sequence-dependent RNA-binding protein.

17. (canceled)

18. The method of claim 9, wherein the binder domain of shuttle-binder effector fusion protein comprises a protein epitope binder, further wherein:
the protein epitope binder is binding to a target protein,
the protein epitope binder comprises a designed ankyrin repeat protein (DARPin), affibody, monobody, single-chain variable fragment (scFv), or heavy chain antibody (HCab), and
the protein epitope binder may cause inhibition or activation of the target protein.

19. (canceled)

20. The method of claim 9, wherein the genetic modification of the lymphocyte cell is transient.

21. The method of claim 9, wherein the genetic modification of the lymphocyte cell is stable.

22. The method of claim 9, wherein contact between the delivery cells and the target cells is mediated via a chimeric-antigen receptor (CAR) that induces the formation of an immunologic synapse and secretion of the shuttle-binder-effector fusion protein.

23. (canceled)

24. A composition for delivery of a shuttle-binder-effector fusion protein to target cells, said composition comprising:
cytotoxic lymphocytes specific for the target cells, said cytotoxic lymphocytes being genetically modified to express the shuttle-binder-effector fusion protein, wherein said fusion protein is sequestered in lytic granules of the cytotoxic lymphocytes, further wherein the shuttle-binder-effector fusion protein is deliverable into the target cell via the granzyme-perforin pathway when the cytotoxic lymphocytes interact with the target cells.

25. The composition of claim 24, wherein the cytotoxic lymphocytes are primary lymphocytes, donor-derived lymphocytes, stem-cell derived lymphocytes, or cell-line derived NK cells, cell-line derived T cells, cell-line derived NKT cells, or cell-line derived macrophages.

26. The composition of claim 24, wherein the cytotoxic lymphocytes are modified to express the fusion protein stably.

27. The composition of claim 24, wherein the cytotoxic lymphocytes are modified to express the fusion protein transiently.

28. The composition of claim 24, wherein the fusion protein comprises a granzyme, a protein epitope binder, and a ubiquitin ligase or protease.

29. The composition of claim 24, wherein the fusion protein consists of a granzyme, a sequence-specific DNA-binding domain or RNA-binding domain, and a nuclease.
